SEQUENCE LISTING (19 sequences)

SEQ ID NO: 1 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - protein, 544 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 3 - protein, 534 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNELINQSLAFINQSDELLHNVNAGKSTTNIMITTIIIVIIVILLSLIAVGLLLYCKARSTPVTLSKDQLSGINNIAFSN

SEQ ID NO: 4 - RNA, 1725 nt, synthetic construct
  source 1..1725: mol_type = other RNA
atggaattgctgatcctcaaagcgaacgcaatcaccactatcctcactgcggtcaccttctgctttgcgagcggacagaacatcaccgaagaattctaccaatctacttgctccgccgtgtccaagggttacctgtccgccctgaggaccggatggtacacttccgtgattaccattgagttgtcgaatatcaagaagaacaagtgcaacggaaccgatgctaaggtcaagctgatcaagcaggagctggacaagtacaagaatgctgtgaccgagctgcagctgctgatgcagtccactcaagccaccaacaatcgcgcccggcgggaactcccaaggttcatgaactacaccttgaacaacgccaagaaaacgaacgtgaccctgtccaagaagcgcaagcgcagattccttggcttccttctgggcgtcggtagcgccatcgcctccggcgtggccgtcagcaaggtcctgcacctcgagggagaagtcaacaagattaagagcgccctgctgtccaccaacaaggccgtggtgtcgctatcaaacggcgtcagcgtactgaccagcaaagtgctggatctcaagaactacattgataagcaactcctccctatcgtgaataagcagagctgttcgatttccaacatcgagactgtgattgaattccagcagaagaacaaccggctgctggaaattaccagagaattcagcgtgaatgccggagtcactacccccgtgtccacctacatgctgacaaactccgagctgctgagcctgatcaacgatatgccgattaccaacgaccagaagaagctgatgtcgaacaacgtgcagatcgtgcgccagcagtcctactcaatcatgtcgatcatcaaggaagaggtcctggcctacgtggtgcagcttcctctgtacggcgtgattgacactccgtgttggaaactgcacactagtcccctgtgcactactaacaccaaggagggcagcaatatctgcctgactcggaccgatagaggctggtactgtgataacgccgggtccgtgtccttcttcccgcaagccgagacttgcaaagtgcagagcaaccgggtgttctgtgacactatgaactcactgaccttgccgagcgaagtcaacctttgcaacgtggacatctttaaccctaaatacgactgcaagatcatgacctccaagaccgacgtgtcgagctcagtgattacttcgctgggagccattgtgtcctgctacgggaaaaccaagtgcacggcctcaaacaagaaccggggtatcattaagaccttctccaacggctgcgactatgtgtccaacaagggggtggacactgtgtccgtgggaaacaccttgtattacgtgaacaagcaggagggaaagtccctctacgtgaagggcgaacccatcatcaatttctacgacccgctcgtgttcccctccgatgaattcgacgcatccatctcacaagtcaacgaaaagattaaccagtccctggctttcattcgcaagtccgacgaactgctccataacgtcaacgctggaaagtccaccaccaacatcatgatcaccacgatcattattgtgatcatcgtcatcctgctgtcactgatagcagtgggactgctcctctactgcaaagcgcggtcgaccccagtgacactctcgaaggaccagctgtccgggatcaacaacatcgcgttttcgaactga

SEQ ID NO: 5 - RNA, 1635 nt, synthetic construct
  source 1..1635: mol_type = other RNA
atggaactcctgatcctgaaggccaatgctatcactaccatcctgactgccgtcaccttctgcttcgcctccggacaaaatatcactgaagaattttaccaaagcacctgtagcgcggtgtccaagggatacctgagcgctctgaggaccggatggtacaccagcgtgattaccatcgagctgagtaacatcaagaagaacaagtgcaacgggaccgatgctaaggtcaagttgatcaaacaagagctcgacaagtacaagaacgccgtgactgagctgcagctgctgatgcagtcaactcaggccaccaacaaccgggccagacgggaactgccgagattcatgaactacaccctgaacaacgccaaaaagaccaacgtgaccctgtccaagaagagaaagcgccggttcctgggtttcctgcttggcgtgggatcagcaatcgcgtccggagtggcagtgtccaaggtcttgcacctcgagggcgaagtgaacaagatcaagtccgcgcttctgtcgaccaacaaggccgtcgtttccctgtcgaacggagtgtccgtgctcacgagcaaagtgctcgacctgaagaactacatcgacaaacagctgctgcccatcgtcaacaagcagagctgcagcatctcaaacattgaaaccgtgatcgagttccagcagaagaacaaccgcctgctcgagattaccagagagttttccgtgaacgccggcgtgaccaccccggtgtcgacctacatgctcacaaattcggaacttctctccctgattaatgacatgcccattactaacgatcagaaaaagctgatgtcgaacaatgtgcagattgtgcgccagcagtcctactccatcatgtccatcattaaggaagaggtcctggcctacgtggtgcagttgccgctgtacggtgtcatcgataccccctgctggaagctccatacttcgcccctgtgtactaccaacaccaaggaaggctccaacatctgcctgacccggacggatcgcggctggtactgtgacaatgccggatccgtgtcgttcttcccgcaagcggagacttgcaaagtgcagtccaaccgggtgttctgtgacactatgaactccctgactctgccgtccgaagtcaacctctgcaacgtggacattttcaatccaaaatacgactgcaagataatgacctccaagactgacgtgtcatcgtccgtgatcacatctctgggagccattgtctcctgctacggaaagactaagtgcaccgcgtcgaacaagaacaggggcattatcaagaccttcagcaacggttgcgactatgtgtccaacaagggcgtggataccgtgtccgtgggcaacaccttgtactacgtgaacaagcaggaggggaagtccctttatgtgaagggggagccaatcattaacttttacgaccccctggtgttccctagcgacgagttcgacgcctcaatctctcaagtcaacgaaaagatcaaccagagcctcgccttcatccgcaagtccgatgaactgctgtcagccattgggggttacatccctgaggcccctcgggacggacaggcatacgtccgcaaggacggcgaatgggtgctgcttagcaccttcctctaa

SEQ ID NO: 6 - RNA, 1605 nt, synthetic construct
  source 1..1605: mol_type = other RNA
atggaactgctgatcctcaaagccaacgcaatcaccaccattctcaccgctgtgaccttctgcttcgcatcggggcagaacatcactgaagagttttaccagagcacttgcagcgcggtgtcaaagggttacctttccgcactgcggaccggatggtacacttccgtgatcaccattgagctcagcaacatcaaggaaaacaagtgcaatggcaccgacgccaaggtcaagctgatcaaacaagaactggacaagtacaagaacgccgtgacagaattgcagctcctgatgggatccggaaacgtcggtctgggcggagccatcgcgagtggagtggctgtgtccaaggtcttgcacctcgagggagaagtgaacaagatcaagtccgcgctgctgtcaacgaacaaggccgtggtgtccctgtctaacggcgtcagcgtgctgacgttcaaggtcctggacctgaagaattacattgacaagcagctgctgcccatcctcaacaagcaatcctgctccatctccaaccccgaaaccgtgatcgagttccagcagaagaacaaccgcctgctggaaattactcgcgagttctctgtgaatgccggcgtgaccacccctgtgtccacctacatgctgaccaactccgagcttctctcccttatcaatgacatgcctatcacgaacgaccagaagaagctgatgtcgaacaacgtgcagattgtgcggcagcagtcatacagcatcatgtcgatcatcaaggaagaagtgctggcgtacgtggtgcaactcccgctgtacggcgtcatcgataccccgtgctggaagctgcacacctcgcctttgtgtaccaccaacaccaagaacggatccaacatctgcttaacccggactgatcggggttggtactgcgacaacgccgggaatgtttcgttcttcccacaagccgagacttgtaaagtgcagtcaaacagagtgttctgtgacaccatgaactcgagaaccctgcccagcgaagtgaacctgtgtaacgtcgacatctttaacccaaaatacgattgcaagattatgaccagcaaaaccgacgtgtcctcctccgtgataacaagcctgggggcgattgtgtcatgctacggaaagactaagtgcaccgcctcgaacaagaaccgcggcatcattaagactttctcgaatggttgcgactatgtgtccaacaagggcgtggatactgtgtcagtcgggaatactctttactacgtgaacaagcaggaggggaaaagcctctacgtgaagggagagcctattatcaacttttacgatccgctggtgttcccgtccgacgaattcgacgccagcatcagccaagtcaacgagctgattaaccagtccctcgccttcatcaaccaatccgacgagctcctgcataacgtgaacgccggaaagtccaccaccaacatcatgatcactactattatcatcgtgatcatcgtcatcctgctgagcctgattgctgtgggcctgttgctgtattgcaaagccaggtccaccccggtcaccctgtcgaaggatcagctgtccggaatcaacaacattgccttctccaactaa

SEQ ID NO: 7 - DNA, 1725 bp, synthetic construct
  source 1..1725: mol_type = other DNA
atggaattgctgatcctcaaagcgaacgcaatcaccactatcctcactgcggtcaccttctgctttgcgagcggacagaacatcaccgaagaattctaccaatctacttgctccgccgtgtccaagggttacctgtccgccctgaggaccggatggtacacttccgtgattaccattgagttgtcgaatatcaagaagaacaagtgcaacggaaccgatgctaaggtcaagctgatcaagcaggagctggacaagtacaagaatgctgtgaccgagctgcagctgctgatgcagtccactcaagccaccaacaatcgcgcccggcgggaactcccaaggttcatgaactacaccttgaacaacgccaagaaaacgaacgtgaccctgtccaagaagcgcaagcgcagattccttggcttccttctgggcgtcggtagcgccatcgcctccggcgtggccgtcagcaaggtcctgcacctcgagggagaagtcaacaagattaagagcgccctgctgtccaccaacaaggccgtggtgtcgctatcaaacggcgtcagcgtactgaccagcaaagtgctggatctcaagaactacattgataagcaactcctccctatcgtgaataagcagagctgttcgatttccaacatcgagactgtgattgaattccagcagaagaacaaccggctgctggaaattaccagagaattcagcgtgaatgccggagtcactacccccgtgtccacctacatgctgacaaactccgagctgctgagcctgatcaacgatatgccgattaccaacgaccagaagaagctgatgtcgaacaacgtgcagatcgtgcgccagcagtcctactcaatcatgtcgatcatcaaggaagaggtcctggcctacgtggtgcagcttcctctgtacggcgtgattgacactccgtgttggaaactgcacactagtcccctgtgcactactaacaccaaggagggcagcaatatctgcctgactcggaccgatagaggctggtactgtgataacgccgggtccgtgtccttcttcccgcaagccgagacttgcaaagtgcagagcaaccgggtgttctgtgacactatgaactcactgaccttgccgagcgaagtcaacctttgcaacgtggacatctttaaccctaaatacgactgcaagatcatgacctccaagaccgacgtgtcgagctcagtgattacttcgctgggagccattgtgtcctgctacgggaaaaccaagtgcacggcctcaaacaagaaccggggtatcattaagaccttctccaacggctgcgactatgtgtccaacaagggggtggacactgtgtccgtgggaaacaccttgtattacgtgaacaagcaggagggaaagtccctctacgtgaagggcgaacccatcatcaatttctacgacccgctcgtgttcccctccgatgaattcgacgcatccatctcacaagtcaacgaaaagattaaccagtccctggctttcattcgcaagtccgacgaactgctccataacgtcaacgctggaaagtccaccaccaacatcatgatcaccacgatcattattgtgatcatcgtcatcctgctgtcactgatagcagtgggactgctcctctactgcaaagcgcggtcgaccccagtgacactctcgaaggaccagctgtccgggatcaacaacatcgcgttttcgaactga

SEQ ID NO: 8 - DNA, 1635 bp, synthetic construct
  source 1..1635: mol_type = other DNA
atggaactcctgatcctgaaggccaatgctatcactaccatcctgactgccgtcaccttctgcttcgcctccggacaaaatatcactgaagaattttaccaaagcacctgtagcgcggtgtccaagggatacctgagcgctctgaggaccggatggtacaccagcgtgattaccatcgagctgagtaacatcaagaagaacaagtgcaacgggaccgatgctaaggtcaagttgatcaaacaagagctcgacaagtacaagaacgccgtgactgagctgcagctgctgatgcagtcaactcaggccaccaacaaccgggccagacgggaactgccgagattcatgaactacaccctgaacaacgccaaaaagaccaacgtgaccctgtccaagaagagaaagcgccggttcctgggtttcctgcttggcgtgggatcagcaatcgcgtccggagtggcagtgtccaaggtcttgcacctcgagggcgaagtgaacaagatcaagtccgcgcttctgtcgaccaacaaggccgtcgtttccctgtcgaacggagtgtccgtgctcacgagcaaagtgctcgacctgaagaactacatcgacaaacagctgctgcccatcgtcaacaagcagagctgcagcatctcaaacattgaaaccgtgatcgagttccagcagaagaacaaccgcctgctcgagattaccagagagttttccgtgaacgccggcgtgaccaccccggtgtcgacctacatgctcacaaattcggaacttctctccctgattaatgacatgcccattactaacgatcagaaaaagctgatgtcgaacaatgtgcagattgtgcgccagcagtcctactccatcatgtccatcattaaggaagaggtcctggcctacgtggtgcagttgccgctgtacggtgtcatcgataccccctgctggaagctccatacttcgcccctgtgtactaccaacaccaaggaaggctccaacatctgcctgacccggacggatcgcggctggtactgtgacaatgccggatccgtgtcgttcttcccgcaagcggagacttgcaaagtgcagtccaaccgggtgttctgtgacactatgaactccctgactctgccgtccgaagtcaacctctgcaacgtggacattttcaatccaaaatacgactgcaagataatgacctccaagactgacgtgtcatcgtccgtgatcacatctctgggagccattgtctcctgctacggaaagactaagtgcaccgcgtcgaacaagaacaggggcattatcaagaccttcagcaacggttgcgactatgtgtccaacaagggcgtggataccgtgtccgtgggcaacaccttgtactacgtgaacaagcaggaggggaagtccctttatgtgaagggggagccaatcattaacttttacgaccccctggtgttccctagcgacgagttcgacgcctcaatctctcaagtcaacgaaaagatcaaccagagcctcgccttcatccgcaagtccgatgaactgctgtcagccattgggggttacatccctgaggcccctcgggacggacaggcatacgtccgcaaggacggcgaatgggtgctgcttagcaccttcctctaa

SEQ ID NO: 9 - DNA, 1605 bp, synthetic construct
  source 1..1605: mol_type = other DNA
atggaactgctgatcctcaaagccaacgcaatcaccaccattctcaccgctgtgaccttctgcttcgcatcggggcagaacatcactgaagagttttaccagagcacttgcagcgcggtgtcaaagggttacctttccgcactgcggaccggatggtacacttccgtgatcaccattgagctcagcaacatcaaggaaaacaagtgcaatggcaccgacgccaaggtcaagctgatcaaacaagaactggacaagtacaagaacgccgtgacagaattgcagctcctgatgggatccggaaacgtcggtctgggcggagccatcgcgagtggagtggctgtgtccaaggtcttgcacctcgagggagaagtgaacaagatcaagtccgcgctgctgtcaacgaacaaggccgtggtgtccctgtctaacggcgtcagcgtgctgacgttcaaggtcctggacctgaagaattacattgacaagcagctgctgcccatcctcaacaagcaatcctgctccatctccaaccccgaaaccgtgatcgagttccagcagaagaacaaccgcctgctggaaattactcgcgagttctctgtgaatgccggcgtgaccacccctgtgtccacctacatgctgaccaactccgagcttctctcccttatcaatgacatgcctatcacgaacgaccagaagaagctgatgtcgaacaacgtgcagattgtgcggcagcagtcatacagcatcatgtcgatcatcaaggaagaagtgctggcgtacgtggtgcaactcccgctgtacggcgtcatcgataccccgtgctggaagctgcacacctcgcctttgtgtaccaccaacaccaagaacggatccaacatctgcttaacccggactgatcggggttggtactgcgacaacgccgggaatgtttcgttcttcccacaagccgagacttgtaaagtgcagtcaaacagagtgttctgtgacaccatgaactcgagaaccctgcccagcgaagtgaacctgtgtaacgtcgacatctttaacccaaaatacgattgcaagattatgaccagcaaaaccgacgtgtcctcctccgtgataacaagcctgggggcgattgtgtcatgctacggaaagactaagtgcaccgcctcgaacaagaaccgcggcatcattaagactttctcgaatggttgcgactatgtgtccaacaagggcgtggatactgtgtcagtcgggaatactctttactacgtgaacaagcaggaggggaaaagcctctacgtgaagggagagcctattatcaacttttacgatccgctggtgttcccgtccgacgaattcgacgccagcatcagccaagtcaacgagctgattaaccagtccctcgccttcatcaaccaatccgacgagctcctgcataacgtgaacgccggaaagtccaccaccaacatcatgatcactactattatcatcgtgatcatcgtcatcctgctgagcctgattgctgtgggcctgttgctgtattgcaaagccaggtccaccccggtcaccctgtcgaaggatcagctgtccggaatcaacaacattgccttctccaactaa

SEQ ID NO: 10 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 11 - RNA, 100 nt, synthetic construct
  source 1..100: mol_type = other RNA
cgggtggcatccctgtgacccctccccagtgcctctcctggccctggaagttgccactccagtgcccaccagccttgtcctaataaaattaagttgcatc

SEQ ID NO: 12 - RNA, 1965 nt, synthetic construct
  source 1..1965: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggaattgctgatcctcaaagcgaacgcaatcaccactatcctcactgcggtcaccttctgctttgcgagcggacagaacatcaccgaagaattctaccaatctacttgctccgccgtgtccaagggttacctgtccgccctgaggaccggatggtacacttccgtgattaccattgagttgtcgaatatcaagaagaacaagtgcaacggaaccgatgctaaggtcaagctgatcaagcaggagctggacaagtacaagaatgctgtgaccgagctgcagctgctgatgcagtccactcaagccaccaacaatcgcgcccggcgggaactcccaaggttcatgaactacaccttgaacaacgccaagaaaacgaacgtgaccctgtccaagaagcgcaagcgcagattccttggcttccttctgggcgtcggtagcgccatcgcctccggcgtggccgtcagcaaggtcctgcacctcgagggagaagtcaacaagattaagagcgccctgctgtccaccaacaaggccgtggtgtcgctatcaaacggcgtcagcgtactgaccagcaaagtgctggatctcaagaactacattgataagcaactcctccctatcgtgaataagcagagctgttcgatttccaacatcgagactgtgattgaattccagcagaagaacaaccggctgctggaaattaccagagaattcagcgtgaatgccggagtcactacccccgtgtccacctacatgctgacaaactccgagctgctgagcctgatcaacgatatgccgattaccaacgaccagaagaagctgatgtcgaacaacgtgcagatcgtgcgccagcagtcctactcaatcatgtcgatcatcaaggaagaggtcctggcctacgtggtgcagcttcctctgtacggcgtgattgacactccgtgttggaaactgcacactagtcccctgtgcactactaacaccaaggagggcagcaatatctgcctgactcggaccgatagaggctggtactgtgataacgccgggtccgtgtccttcttcccgcaagccgagacttgcaaagtgcagagcaaccgggtgttctgtgacactatgaactcactgaccttgccgagcgaagtcaacctttgcaacgtggacatctttaaccctaaatacgactgcaagatcatgacctccaagaccgacgtgtcgagctcagtgattacttcgctgggagccattgtgtcctgctacgggaaaaccaagtgcacggcctcaaacaagaaccggggtatcattaagaccttctccaacggctgcgactatgtgtccaacaagggggtggacactgtgtccgtgggaaacaccttgtattacgtgaacaagcaggagggaaagtccctctacgtgaagggcgaacccatcatcaatttctacgacccgctcgtgttcccctccgatgaattcgacgcatccatctcacaagtcaacgaaaagattaaccagtccctggctttcattcgcaagtccgacgaactgctccataacgtcaacgctggaaagtccaccaccaacatcatgatcaccacgatcattattgtgatcatcgtcatcctgctgtcactgatagcagtgggactgctcctctactgcaaagcgcggtcgaccccagtgacactctcgaaggaccagctgtccgggatcaacaacatcgcgttttcgaactgacgggtggcatccctgtgacccctccccagtgcctctcctggccctggaagttgccactccagtgcccaccagccttgtcctaataaaattaagttgcatc

SEQ ID NO: 13 - RNA, 1875 nt, synthetic construct
  source 1..1875: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggaactcctgatcctgaaggccaatgctatcactaccatcctgactgccgtcaccttctgcttcgcctccggacaaaatatcactgaagaattttaccaaagcacctgtagcgcggtgtccaagggatacctgagcgctctgaggaccggatggtacaccagcgtgattaccatcgagctgagtaacatcaagaagaacaagtgcaacgggaccgatgctaaggtcaagttgatcaaacaagagctcgacaagtacaagaacgccgtgactgagctgcagctgctgatgcagtcaactcaggccaccaacaaccgggccagacgggaactgccgagattcatgaactacaccctgaacaacgccaaaaagaccaacgtgaccctgtccaagaagagaaagcgccggttcctgggtttcctgcttggcgtgggatcagcaatcgcgtccggagtggcagtgtccaaggtcttgcacctcgagggcgaagtgaacaagatcaagtccgcgcttctgtcgaccaacaaggccgtcgtttccctgtcgaacggagtgtccgtgctcacgagcaaagtgctcgacctgaagaactacatcgacaaacagctgctgcccatcgtcaacaagcagagctgcagcatctcaaacattgaaaccgtgatcgagttccagcagaagaacaaccgcctgctcgagattaccagagagttttccgtgaacgccggcgtgaccaccccggtgtcgacctacatgctcacaaattcggaacttctctccctgattaatgacatgcccattactaacgatcagaaaaagctgatgtcgaacaatgtgcagattgtgcgccagcagtcctactccatcatgtccatcattaaggaagaggtcctggcctacgtggtgcagttgccgctgtacggtgtcatcgataccccctgctggaagctccatacttcgcccctgtgtactaccaacaccaaggaaggctccaacatctgcctgacccggacggatcgcggctggtactgtgacaatgccggatccgtgtcgttcttcccgcaagcggagacttgcaaagtgcagtccaaccgggtgttctgtgacactatgaactccctgactctgccgtccgaagtcaacctctgcaacgtggacattttcaatccaaaatacgactgcaagataatgacctccaagactgacgtgtcatcgtccgtgatcacatctctgggagccattgtctcctgctacggaaagactaagtgcaccgcgtcgaacaagaacaggggcattatcaagaccttcagcaacggttgcgactatgtgtccaacaagggcgtggataccgtgtccgtgggcaacaccttgtactacgtgaacaagcaggaggggaagtccctttatgtgaagggggagccaatcattaacttttacgaccccctggtgttccctagcgacgagttcgacgcctcaatctctcaagtcaacgaaaagatcaaccagagcctcgccttcatccgcaagtccgatgaactgctgtcagccattgggggttacatccctgaggcccctcgggacggacaggcatacgtccgcaaggacggcgaatgggtgctgcttagcaccttcctctaacgggtggcatccctgtgacccctccccagtgcctctcctggccctggaagttgccactccagtgcccaccagccttgtcctaataaaattaagttgcatc

SEQ ID NO: 14 - RNA, 1845 nt, synthetic construct
  source 1..1845: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggaactgctgatcctcaaagccaacgcaatcaccaccattctcaccgctgtgaccttctgcttcgcatcggggcagaacatcactgaagagttttaccagagcacttgcagcgcggtgtcaaagggttacctttccgcactgcggaccggatggtacacttccgtgatcaccattgagctcagcaacatcaaggaaaacaagtgcaatggcaccgacgccaaggtcaagctgatcaaacaagaactggacaagtacaagaacgccgtgacagaattgcagctcctgatgggatccggaaacgtcggtctgggcggagccatcgcgagtggagtggctgtgtccaaggtcttgcacctcgagggagaagtgaacaagatcaagtccgcgctgctgtcaacgaacaaggccgtggtgtccctgtctaacggcgtcagcgtgctgacgttcaaggtcctggacctgaagaattacattgacaagcagctgctgcccatcctcaacaagcaatcctgctccatctccaaccccgaaaccgtgatcgagttccagcagaagaacaaccgcctgctggaaattactcgcgagttctctgtgaatgccggcgtgaccacccctgtgtccacctacatgctgaccaactccgagcttctctcccttatcaatgacatgcctatcacgaacgaccagaagaagctgatgtcgaacaacgtgcagattgtgcggcagcagtcatacagcatcatgtcgatcatcaaggaagaagtgctggcgtacgtggtgcaactcccgctgtacggcgtcatcgataccccgtgctggaagctgcacacctcgcctttgtgtaccaccaacaccaagaacggatccaacatctgcttaacccggactgatcggggttggtactgcgacaacgccgggaatgtttcgttcttcccacaagccgagacttgtaaagtgcagtcaaacagagtgttctgtgacaccatgaactcgagaaccctgcccagcgaagtgaacctgtgtaacgtcgacatctttaacccaaaatacgattgcaagattatgaccagcaaaaccgacgtgtcctcctccgtgataacaagcctgggggcgattgtgtcatgctacggaaagactaagtgcaccgcctcgaacaagaaccgcggcatcattaagactttctcgaatggttgcgactatgtgtccaacaagggcgtggatactgtgtcagtcgggaatactctttactacgtgaacaagcaggaggggaaaagcctctacgtgaagggagagcctattatcaacttttacgatccgctggtgttcccgtccgacgaattcgacgccagcatcagccaagtcaacgagctgattaaccagtccctcgccttcatcaaccaatccgacgagctcctgcataacgtgaacgccggaaagtccaccaccaacatcatgatcactactattatcatcgtgatcatcgtcatcctgctgagcctgattgctgtgggcctgttgctgtattgcaaagccaggtccaccccggtcaccctgtcgaaggatcagctgtccggaatcaacaacattgccttctccaactaacgggtggcatccctgtgacccctccccagtgcctctcctggccctggaagttgccactccagtgcccaccagccttgtcctaataaaattaagttgcatc

SEQ ID NO: 15 - protein, 7 aa, synthetic construct
GSGNVGL

SEQ ID NO: 16 - protein, 9 aa, synthetic construct
GSGNVGLGG

SEQ ID NO: 17 - protein, 50 aa, synthetic construct
IMITTIIIVIIVILLSLIAVGLLLYCKARSTPVTLSKDQLSGINNIAFSN

SEQ ID NO: 18 - RNA, 11 nt, synthetic construct
  source 1..11: mol_type = other RNA
gggatcctacc

SEQ ID NO: 19 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2023/0302112 A1
(43) Pub. Date: Sep. 28, 2023

(54) RESPIRATORY SYNCTIAL VIRUS RNA VACCINE

(71) Applicant: SANOFI, Paris (FR)

(72) Inventors: Danilo Casimiro, Harleysville, PA (US); Hardip Rajeshbhai Gopani, Woburn, MA (US); Joshua DiNapoli, Lexington, MA (US); Linong Zhang, Braintree, MA (US); Mark Parrington, Holbrook, MA (US); Rebecca L. Goldman, Weston, MA (US); Sudha Chivukula, Cambridge, MA (US); William Scott Gallichan, Burlington (CA)

(21) Appl. No.: 18/052,600

(22) Filed: Nov. 4, 2022

Related U.S. Application Data
(60) Provisional application No. 63/276,233, filed on Nov. 5, 2021.

(30) Foreign Application Priority Data
Mar. 16, 2022 (EP) 22315065.7

(51) Int. Cl. A61K 39/12; C07K 14/135; A61P 31/14

(52) U.S. Cl. CPC A61K 39/12; C07K 14/135; A61P 31/14; A61K 2039/55555

(57) ABSTRACT

The present disclosure provides a respiratory syncytial virus (RSV) vaccine comprising a messenger RNA (mRNA) comprising an open reading frame (ORF) encoding an RSV F protein antigen, and methods of eliciting an immune response by administering said vaccine.

### RELATED APPLICATIONS

This application claims the benefit of U.S. Provisional Application No. 63/276,233, filed on Nov. 5, 2021, and European Patent Application No. 22315065.7, filed on Mar. 16, 2022, each of which is incorporated by reference in its entirety for all purposes.

### BACKGROUND OF THE DISCLOSURE

Respiratory syncytial virus (RSV) is a leading cause of severe respiratory disease in infants and a major cause of respiratory illness in the elderly. RSV remains an unmet vaccine need despite decades of research. Recent clinical programs using an RSV F antigen in its post-fusion conformation failed to elicit sufficient efficacy in adults. See, Faloon et al. (2017) JID 216: 1362-1370. However, RSV F antigens stabilized in the pre-fusion conformation may elicit a neutralizing response superior to that of the post-fusion antigens that have failed in the clinic.

RNA-based vaccines (e.g., mRNA vaccines) have recently emerged as an effective vaccine type against severe acute respiratory syndrome coronavirus 2 (SARS-CoV-2). Coronavirus disease 2019 (COVID-19) mRNA vaccines have exhibited rapid, safe, and cost-effective production processes. Often combined with a delivery vehicle, such as a lipid nanoparticle (LNP), COVID-19 mRNA vaccines can achieve high efficacy. With the dearth of effective RSV vaccines available, there exists a need for RNA-based RSV vaccines that elicit strong immune responses against the RSV pre-fusion F protein for potent neutralization of an RSV infection.

### BRIEF SUMMARY OF THE DISCLOSURE

In one aspect, the disclosure provides a respiratory syncytial virus (RSV) vaccine, comprising a messenger RNA (mRNA) comprising an open reading frame (ORF) encoding an RSV F protein antigen, wherein the RSV F protein antigen comprises an amino acid sequence with at least 98% identity (e.g., 98%, 99%, or 100% identity) to SEQ ID NO: 3 or consists of an amino acid sequence of SEQ ID NO: 3.

In certain embodiments, the RSV F protein antigen is a pre-fusion protein.

In certain embodiments, the ORF is codon optimized.

In certain embodiments, the mRNA comprises at least one 5′ untranslated region (5′ UTR), at least one 3′ untranslated region (3′ UTR), and at least one polyadenylation (poly(A)) sequence.

In certain embodiments, the mRNA comprises at least one chemical modification.

In certain embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In certain embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

In certain embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In certain embodiments, the chemical modification is selected from the group consisting of pseudouridine, N1-methylpseudouridine, 5-methylcytosine, 5-methoxyuridine, and a combination thereof.

In certain embodiments, the chemical modification is N1-methylpseudouridine.

In certain embodiments, the mRNA is formulated in a lipid nanoparticle (LNP).

In certain embodiments, the LNP comprises at least one cationic lipid.

In certain embodiments, the cationic lipid is biodegradable. In certain embodiments, the cationic lipid is not biodegradable.

In certain embodiments, the cationic lipid is cleavable. In certain embodiments, the cationic lipid is not cleavable.

In certain embodiments, the cationic lipid is selected from the group consisting of OF-02, cKK-E10, GL-HEPES-E3-E10-DS-3-E18-1, GL-HEPES-E3-E12-DS-4-E10, and GL-HEPES-E3-E12-DS-3-E14.

In certain embodiments, the cationic lipid is cKK-E10.

In certain embodiments, the cationic lipid is GL-HEPES-E3-E12-DS-4-E10.

In certain embodiments, the LNP further comprises a polyethylene glycol (PEG) conjugated (PEGylated) lipid, a cholesterol-based lipid, and a helper lipid.

In certain embodiments, the LNP comprises: a cationic lipid at a molar ratio of 35% to 55%; a polyethylene glycol (PEG) conjugated (PEGylated) lipid at a molar ratio of 0.25% to 2.75%; a cholesterol-based lipid at a molar ratio of 20% to 45%; and a helper lipid at a molar ratio of 5% to 35%, wherein all of the molar ratios are relative to the total lipid content of the LNP.

In certain embodiments, the LNP comprises: a cationic lipid at a molar ratio of 40%, a PEGylated lipid at a molar ratio of 1.5%, a cholesterol-based lipid at a molar ratio of 28.5%, and a helper lipid at a molar ratio of 30%.

In certain embodiments, the PEGylated lipid is dimyristoyl-PEG2000 (DMG-PEG2000) or 2-[(polyethylene glycol)-2000]-N,N-ditetradecylacetamide (ALC-0159).

In certain embodiments, the cholesterol-based lipid is cholesterol.

In certain embodiments, the helper lipid is 1,2-dioleoyl-SN-glycero-3-phosphoethanolamine (DOPE) or 1,2-distearoyl-sn-glycero-3-phosphocholine (DSPC).

In certain embodiments, the LNP comprises: GL-HEPES-E3-E12-DS-4-E10 at a molar ratio of 40%, DMG-PEG2000 at a molar ratio of 1.5%, cholesterol at a molar ratio of 28.5%, and DOPE at a molar ratio of 30%.

In certain embodiments, the LNP comprises: cKK-E10 at a molar ratio of 40%, DMG-PEG2000 at a molar ratio of 1.5%, cholesterol at a molar ratio of 28.5%, and DOPE at a molar ratio of 30%.

In certain embodiments, the LNP has an average diameter of 30 nm to 200 nm. In certain embodiments, the LNP has an average diameter of 80 nm to 150 nm.

In certain embodiments, the mRNA comprises a nucleic acid sequence with at least 80% identity to a nucleic acid sequence set forth in SEQ ID NO: 6.

In certain embodiments, the mRNA comprises a nucleic acid sequence with at least 80% identity to a nucleic acid sequence set forth in SEQ ID NO: 14.

In certain embodiments, the mRNA comprises of the following structural elements:
(i) a 5′ cap with the following structure:

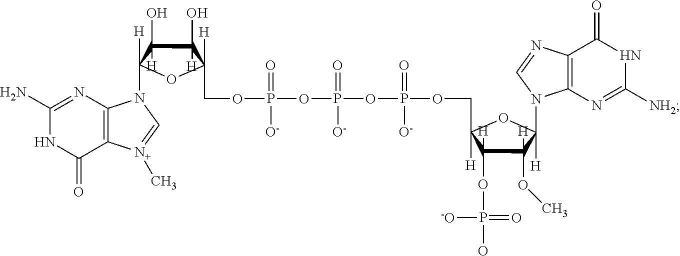

(ii) a 5′ untranslated region (5′ UTR) having the nucleic acid sequence of SEQ ID NO: 10;
(iii) a protein coding region having the nucleic acid sequence of SEQ ID NO: 6;
(iv) a 3′ untranslated region (3′ UTR) having the nucleic acid sequence of SEQ ID NO: 11; and
(v) a poly(A) tail.

In one aspect, the disclosure provides a respiratory syncytial virus (RSV) vaccine, comprising a messenger RNA (mRNA) comprising an open reading frame (ORF) encoding an RSV F protein antigen, wherein the mRNA comprises of the following structural elements:
(i) a 5′ cap with the following structure:

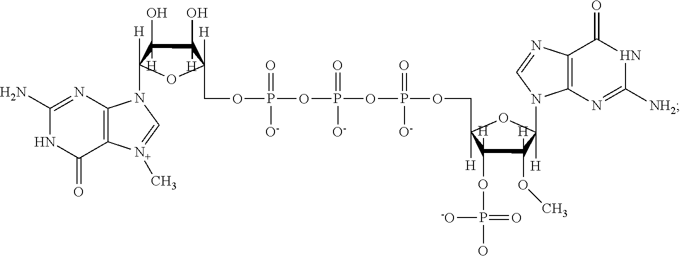

(ii) a 5′ untranslated region (5′ UTR) having the nucleic acid sequence of SEQ ID NO: 10; (iii) a protein coding region having the nucleic acid sequence of SEQ ID NO: 6;
(iv) a 3′ untranslated region (3′ UTR) having the nucleic acid sequence of SEQ ID NO: 11; and (v) a poly(A) tail;
wherein the mRNA is formulated in a lipid nanoparticle (LNP) comprising: GL-HEPES-E3-E12-DS-4-E10 at a molar ratio of 40%, DMG-PEG2000 at a molar ratio of 1.5%, cholesterol at a molar ratio of 28.5%, and DOPE at a molar ratio of 30%.

In one aspect, the disclosure provides a respiratory syncytial virus (RSV) vaccine, comprising a messenger RNA (mRNA) comprising an open reading frame (ORF) encoding an RSV F protein antigen, wherein the mRNA comprises of the following structural elements:
(i) a 5′ cap with the following structure:

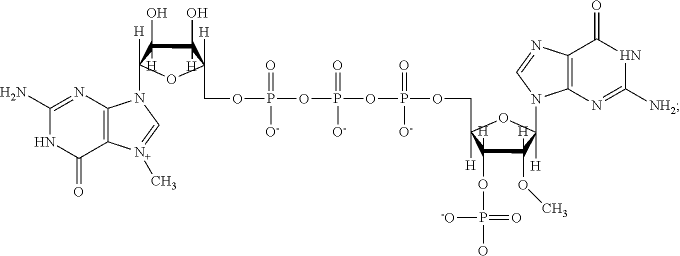

(ii) a 5′ untranslated region (5′ UTR) having the nucleic acid sequence of SEQ ID NO: 10;
(iii) a protein coding region having the nucleic acid sequence of SEQ ID NO: 6;
(iv) a 3′ untranslated region (3′ UTR) having the nucleic acid sequence of SEQ ID NO: 11; and
(v) a poly(A) tail;
wherein the mRNA is formulated in a lipid nanoparticle (LNP) comprising: cKK-E10 at a molar ratio of 40%, DMG-PEG2000 at a molar ratio of 1.5%, cholesterol at a molar ratio of 28.5%, and DOPE at a molar ratio of 30%.

In another aspect, the disclosure provides a method of eliciting an immune response to RSV or protecting a subject against RSV infection, comprising administering the RSV vaccine described above to a subject.

In certain embodiments, the subject has a higher serum concentration of neutralizing antibodies against RSV after administration of the RSV vaccine, relative to a subject that is administered an RSV vaccine comprising an mRNA ORF encoding an RSV F protein antigen of SEQ ID NO: 1.

In certain embodiments, the subject has a comparable serum concentration of neutralizing antibodies against RSV after administration of the RSV vaccine, relative to a subject that is administered a protein RSV vaccine.

In certain embodiments, the protein RSV vaccine is co-administered with an adjuvant.

In certain embodiments, the RSV vaccine increases the serum concentration of antibodies with binding specificity to site Ø of the RSV F protein.

In certain embodiments, the subject has a lower serum concentration of antibodies with binding specificity to site I or site II of the RSV F protein after administration of the RSV vaccine, relative to a subject that is administered an RSV vaccine comprising an mRNA ORF encoding an RSV F protein antigen of SEQ ID NO: 2.

In certain embodiments, the RSV vaccine increases the serum concentration of neutralizing antibodies in a subject with pre-existing RSV immunity.

In another aspect, the disclosure provides an RSV vaccine for use in eliciting an immune response to RSV or protecting a subject against RSV infection, comprising administering the RSV vaccine described above to a subject.

In certain embodiments, the RSV vaccine described above is used in the manufacture of a medicament for eliciting an immune response to RSV or protecting a subject against RSV infection.

### BRIEF DESCRIPTION OF THE DRAWINGS/FIGURES

The foregoing and other features and advantages of the present disclosure will be more fully understood from the following detailed description of illustrative embodiments taken in conjunction with the accompanying drawings.

FIG. 1A-FIG. 1B depict western blot images of FD1, FD2, and FD3 proteins from transfected cells. HEK293FT cells seeded in 6-well plates were transfected with 3 μg of mRNA using a MIRUS kit and cell lysates (FD1 and FD3) or cell supernatants (FD2) and were recovered 24-hours post-transfection (FIG. 1A). FD1 mRNA was evaluated by cell-free means using an in vitro transcription (IVT) kit to produce proteins and compared against proteins from FD1 mRNA transfected cells. Recovered samples were run for western blot analysis and membranes stained with 5353C75 monoclonal antibody (FIG. 1B).

FIG. 2A-FIG. 2B depict western blots of transfected HEKs vs. nucleofected HSkMCs. HEK293FT cells seeded in 6-well plates were transfected with 5 μg of mRNA using a MIRUS kit and cell lysates (FD1 and FD3) or cell supernatants (FD2) were recovered 24-hours post-transfection (FIG. 2A). HSkM cells were nucleofected with 5 μg of mRNA using an Amaxa basic nucleofector kit and cell lysates (FD1 and FD3) or cell supernatants (FD2) were recovered 24-hours post-nucleofection (FIG. 2B). Recovered samples were run for western blot analysis and membranes stained with 5353C75 monoclonal antibody.

FIG. 3 depicts immunostaining of transfected HEK cells. HEK293FT cells seeded in 24-well plates were transfected with 5 μg of mRNA using MIRUS kit cells. Twenty-four hours post-transfection, monoclonal antibodies D25 and Synagis were added to the plates with a fluorescently tagged secondary antibody and imaged using a Celigo.

FIG. 4A-FIG. 4B depict the immunogenicity of selected RSV antigens in naïve non-human primates (NHP). The RSV F protein antibody titer (FIG. 4A) and RSV microneutralization titer (FIG. 4B) were measured at day 0, 28, and 56 for each antigenic composition.

FIG. 5 depicts the results of a competitive ELISA with serum of NHPs immunized with selected RSV antigens against three known RSV F protein antibodies, D25, Synagis (palivizumab), and 131-2a.

FIG. 6A-FIG. 6B depict pre-immune boosting effects in cynomolgus macaques by RSV F ELISA and RSV microneutralization assay. Titers of anti-RSV-F antibodies in boosted monkeys were measured by end-point ELISA using the DS-Cav1 Pre-F protein as the binding antigen and detected with goat-anti-human IgG. Readouts from individual animals (n=6) are shown for DO, D14, and D28 timepoints with GMT+1-95% confidence interval (value above each=GMT). Statistical analysis was performed using two-way ANOVA with Tukey's post-hoc test for multiple comparisons (FIG. 6A). RSV neutralizing antibody titers were measured by a microneutralization assay using a WT A2-GFP RSV strain mixed with serially diluted sera of vaccinated monkeys on 96-well plates of Vero cells. Titers were determined by calculating the inverse reduction of fluorescent foci after a 24-hour incubation. Readouts from individual animals (n=6) are shown for DO, D14, and D28 timepoints with GMT+/−95% confidence interval (value above each=GMT). Statistical analysis was performed using two-way ANOVA with Tukey's post-hoc test for multiple comparisons (FIG. 6B).

FIG. 7 depicts RSV F protein antibody titers in NHPs immunized with the FD3 F protein expressing mRNA. The mRNA was delivered with lipid nanoparticles (LNPs) containing one of several cationic lipids. The antibody titers were measured at day 0, 21, and 35 for each antigenic composition.

FIG. 8 depicts RSV neutralization titers in NHPs immunized with the FD3 F protein expressing mRNA. The mRNA was delivered with lipid nanoparticles (LNPs) containing one of several cationic lipids. The antibody titers were measured at day 0, 21, and 35 for each antigenic composition.

FIG. 9A-FIG. 9B depict the immunogenicity of selected RSV antigens in naïve mice. The RSV F protein antibody titer (FIG. 9A) and RSV microneutralization titer (FIG. 9B) were measured at day 0, 21, and 35 for each antigenic composition.

FIG. 10A-FIG. 10B depict the Pre-F IgG titer (FIG. 10A) and Pre-F/Post-F binding ratio (FIG. 10B) with selected RSV antigens in the modular immune in vitro construct (MIMIC®) system.

FIG. 11 depicts the anti-RSV neutralization titer in the MIMIC® system derived from donors with pre-existing RSV immunity.

### DETAILED DESCRIPTION OF THE DISCLOSURE

The present disclosure is directed to, inter alia, novel RNA (e.g., mRNA) compositions encoding an RSV F protein and methods of vaccination with the same. In particular, the disclosure relates to mRNA encoding an RSV Pre-F protein formulated in a lipid nanoparticle (LNP).

### I. Definitions

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, protein and nucleic acid chemistry, and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, may provide one of skill with a general dictionary of many of the terms used in this disclosure.

Units, prefixes, and symbols are denoted in their International System of Units (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

As used herein, the term “messenger RNA” or “mRNA” refers to a polynucleotide that encodes at least one polypeptide. mRNA, as used herein, encompasses both modified and unmodified RNA. mRNA may contain one or more coding and non-coding regions. A coding region is alternatively referred to as an open reading frame (ORF). Non-coding regions in mRNA include the 5′ cap, 5′ untranslated region (UTR), 3′ UTR, and a poly(A) tail. mRNA can be purified from natural sources, produced using recombinant expression systems (e.g., in vitro transcription) and optionally purified or chemically synthesized.

As used herein, the term “antigenic site Ø” or “site Ø epitope” refers to a site located at the apex of the pre-fusion RSV F trimer, comprising amino acid residues 62-69 and 196-209 of wild-type RSV F (SEQ ID NO: 1). The site Ø epitope is a binding site for antibodies that have specificity for pre-fusion RSV F, such as D25 and AM14, and binding of antibodies to the site Ø epitope blocks cell-surface attachment of RSV (see, e.g., McLellan et al., Science, 340(6136): 1113-1117, 2013). Recombinant human anti-RSV antibody D25 (Creative Biolabs®; CAT #: PABL-322) and recombinant human anti-RSV antibody AM14 (Creative Biolabs®; CAT #: PABL-321) are each commercially available.

As used herein, the term “antigen stability” refers to stability of the antigen over time or in solution.

As used herein, the term “cavity filling substitutions” refers to engineered hydrophobic substitutions to fill cavities present in the pre-fusion RSV F trimer.

As used herein, the term “F protein” or “RSV F protein” refers to the protein of RSV responsible for driving fusion of the viral envelope with host cell membrane during viral entry.

As used herein, the term “RSV F polypeptide” or “F polypeptide” refers to a polypeptide comprising at least one epitope of F protein.

As used herein, the term “glycan addition” refers to the addition of mutations which introduce glycosylation sites not present in wild-type RSV F, which can be engineered to increase construct expression, increase construct stability, or block epitopes shared between the pre-fusion and post-fusion conformation. A modified protein comprising glycan additions would have more glycosylation and therefore a higher molecular weight. Glycan addition can reduce the extent to which an RSV F polypeptide elicits antibodies to the post-fusion conformation of RSV F.

As used herein, the term “intra-protomer stabilizing substitutions” refers to amino acid substitutions in RSV F that stabilize the pre-fusion conformation by stabilizing the interaction within a protomer of the RSV F trimer.

As used herein, the term “inter-protomer stabilizing substitutions” refers to amino acid substitutions in RSV F that stabilize the pre-fusion conformation by stabilizing the interaction of the protomers of the RSV F trimer with each other.

As used herein, the term “protease cleavage” refers to proteolysis (sometimes also referred to as “clipping”) of susceptible residues (e.g., lysine or arginine) in a polypeptide sequence.

As used herein, the term “post-fusion” with respect to RSV F refers to a stable conformation of RSV F that occurs after merging of the virus and cell membranes.

As used herein, the term “pre-fusion” with respect to RSV F refers to a conformation of RSV F that is adopted before virus-cell interaction.

As used herein, the term “protomer” refers to a structural unit of an oligomeric protein. In the case of RSV F, an individual unit of the RSV F trimer is a protomer.

As used herein, the term “N-glycan” refers to a saccharide chain attached to a protein at the amide nitrogen of an N (asparagine) residue of the protein. As such, an N-glycan is formed by the process of N-glycosylation. This glycan may be a polysaccharide.

As used herein, the term “glycosylation” refers to the addition of a saccharide unit to a protein.

As used herein, the term “immune response” refers to a response of a cell of the immune system, such as a B cell, T cell, dendritic cell, macrophage, or polymorphonucleocyte, to a stimulus such as an antigen or vaccine. An immune response can include any cell of the body involved in a host defense response, including, for example, an epithelial cell that secretes an interferon or a cytokine. An immune response includes, but is not limited to, an innate and/or adaptive immune response.

As used herein, a “protective immune response” refers to an immune response that protects a subject from infection (e.g., prevents infection or prevents the development of disease associated with infection). Methods of measuring immune responses include measuring, for example, proliferation and/or activity of lymphocytes (such as B or T cells), secretion of cytokines or chemokines, inflammation, antibody production, and the like.

As used herein, an “antibody response” is an immune response in which antibodies are produced.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity. Antigens include the RSV polypeptides encoded by the mRNA as described herein.

As used herein, an “adjuvant” refers to a substance or vehicle that enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes, killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-Like Receptor (TLR) agonists and costimulatory molecules.

As used herein, an “antigenic RSV polypeptide” refers to a polypeptide comprising all or part of an RSV amino acid sequence of sufficient length that the molecule is antigenic with respect to RSV.

As used herein, a “subject” refers to any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically engineered animal, and/or a clone. In certain embodiments, the subject is an adult, an adolescent, or an infant. In some embodiments, the terms “individual” or “patient” are used and are intended to be interchangeable with “subject.” In certain exemplary embodiments, the subject is a preterm newborn infant (e.g., gestational age less than 37 weeks), a newborn (e.g., 0-27 days of age), an infant or toddler (e.g., 28 days to 23 months of age), a child (e.g., 2 to 11 years of age), an adolescent (e.g., 12 to 17 years of age), an adult (e.g., 18 to 50 years of age or 18 to 64 years of age), or an elderly person (e.g., 65 years of age or older). In exemplary embodiments, the subject is 18 to 50 years of age. In other exemplary embodiments, the subject is an older adult (e.g., an adult aged 60 years of age or older).

As used herein, the term “vaccination” or “vaccinate” refers to the administration of a composition intended to generate an immune response, for example, to a disease-causing agent. Vaccination can be administered before, during, and/or after exposure to a disease-causing agent, and/or to the development of one or more symptoms, and in some embodiments, before, during, and/or shortly after exposure to the disease-causing agent. In some embodiments, vaccination includes multiple administrations, appropriately spaced in time, of a vaccinating composition.

The disclosure describes nucleic acid sequences (e.g., DNA and RNA sequences) and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90%, or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments, in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. In some embodiments, a nucleic acid sequence or amino acid sequence having a particular degree of identity to a given nucleic acid sequence or amino acid sequence is functionally equivalent to said given sequence.

As used herein, the term “kit” refers to a packaged set of related components, such as one or more compounds or compositions and one or more related materials such as solvents, solutions, buffers, instructions, or desiccants.

### II. RNA

The RSV vaccines of the present disclosure may comprise at least one ribonucleic acid (RNA) comprising an ORF encoding an RSV F protein antigen. In certain embodiments, the RNA is a messenger RNA (mRNA) comprising an ORF encoding an RSV F protein antigen. In certain embodiments, the RNA (e.g., mRNA) further comprises at least one 5′ UTR, 3′ UTR, a poly(A) tail, and/or a 5′ cap.

II. A. 5′ Cap

An mRNA 5′ cap can provide resistance to nucleases found in most eukaryotic cells and promote translation efficiency. Several types of 5′ caps are known. A 7-methylguanosine cap (also referred to as “m7G” or “Cap-0”), comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5 ′5 ′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; Tri Link Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

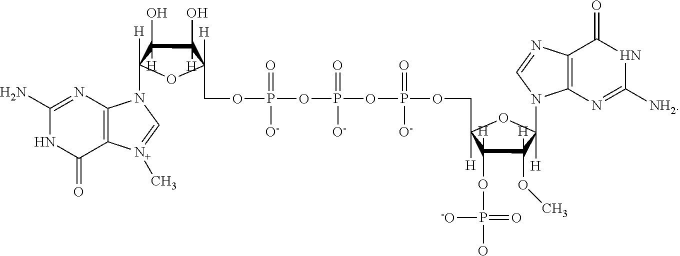

II. B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 18) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′UTR comprises a nucleic acid sequence set forth in SEQ ID NO: 10. In some embodiments, the 3′UTR comprises a nucleic acid sequence set forth in SEQ ID NO: 11. The 5′ UTR and 3′UTR are described in further detail in WO2012/075040, incorporated herein by reference.

II. C. Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence AAAAAAAAAAAAAAAAAAAAAAAAAAAAAAGCAUAUGACUAAAAAAAAAAAAAAAAAAAAAAAAA AAAAAAAAAAAAAAAAAAAAAAAAAAAAAAAAAAAAAAAAAAAAA (SEQ ID NO: 19).

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A)polymerases, e.g., using methods and means as described in WO2016/174271.

The nucleic acid may comprise a poly(A) tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in WO2016/091391.

In certain embodiments, the nucleic acid comprises at least one polyadenylation signal.

In various embodiments, the nucleic acid may comprise at least one poly(C) sequence.

The term “poly(C) sequence,” as used herein, is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In some embodiments, the poly(C) sequence comprises about 30 cytosine nucleotides.

II. D. Chemical Modification

The mRNA disclosed herein may be modified or unmodified. In some embodiments, the mRNA may comprise at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

II. E. mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25(2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

In certain embodiment, the mRNA comprises of the following structural elements:
(i) a 5′ cap with the following structure:

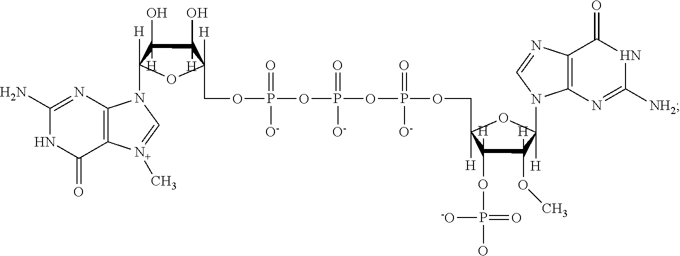

(ii) a 5′ untranslated region (5′ UTR) having the nucleic acid sequence of SEQ ID NO: 10;
(iii) a protein coding region having the nucleic acid sequence of SEQ ID NO: 6;
(iv) a 3′ untranslated region (3′ UTR) having the nucleic acid sequence of SEQ ID NO: 11; and
(v) a poly(A) tail.

In certain embodiments, the poly(A) tail has a length of about 10 to about 500 adenosine nucleotides.

### III. RSV F Protein

Respiratory syncytial virus (RSV) is a negative-sense, single-stranded RNA virus belonging to the Pneumoviridae family. RSV can cause infection of the respiratory tract. RSV is an enveloped virus with a glycoprotein (G protein), small hydrophobic protein (SH protein), and a fusion protein (F protein) on the surface.

The RSV F protein is responsible for fusion of viral and host cell membranes and takes on at least three conformations (pre-fusion, intermediate, and post-fusion conformations). In the pre-fusion conformation (pre-fusion, Pre-F), the F protein exists in a trimeric form with the major antigenic site Ø exposed. Site Ø serves as a primary target of neutralizing antibodies produced by RSV-infected subjects (see, Coultas et al., Thorax. 74: 986-993. 2019; McLellan et al., Science. 340(6136): 1113-7. 2013). After binding to its target on the host cell surface, Pre-F undergoes a conformational change during which site Ø is no longer exposed. Pre-F transitions into a transient intermediate conformation, enabling the F protein to insert into the host cell membrane, leading to fusion of the viral and host cell membranes. A final conformational shift results in a more stable and elongated form of the protein (post-fusion, Post-F). Site II and Site IV of the F protein are specific to Post-F, while Site I is present in both the Pre-F and Post-F conformations (McLellan et al., J. Virol. 85(15): 7788-7796. 2011).

Provided herein are RNAs (e.g., mRNAs) that encode for antigenic RSV F polypeptides.

In one aspect, the disclosure provides a respiratory syncytial virus (RSV) vaccine comprising a messenger RNA (mRNA) comprising an open reading frame (ORF) encoding an RSV F protein antigen, wherein the RSV F protein antigen comprises an amino acid sequence with at least 98% identity to SEQ ID NO: 3 or consists of an amino acid sequence of SEQ ID NO: 3.

In some embodiments, the ORF is codon optimized. As used herein, “codon optimized” or “codon optimization” refers to the introduction of certain codons (in exchange for the respective wild-type codons encoding the same amino acid), which may be more favorable with respect to stability of RNA and/or with respect to codon usage in a subject.

In some embodiments, an epitope of the RSV F protein that is shared between Pre-F and Post-F is blocked. Blocking an epitope reduces or eliminates the generation of antibodies against the epitope when the RNA (e.g., mRNA) that encodes for the antigenic RSV F polypeptide is administered to a subject. This can increase the proportion of antibodies that target an epitope specific to a particular conformation of F, such as the pre-fusion conformation (e.g., antibodies that target site Ø). Because F has the pre-fusion conformation in viruses that have not yet entered cells, an increased proportion of antibodies that target Pre-F can provide a greater degree of neutralization (e.g., expressed as a neutralizing to binding ratio, as described herein). Blocking can be achieved by engineering a bulky moiety such as an N-glycan in the vicinity of the shared epitope. For example, an N-glycosylation site not present in wild-type F can be added, e.g., by mutating an appropriate residue to asparagine. In some embodiments, the blocked epitope is an epitope of antigenic site I of RSV F. In some embodiments, two or more epitopes shared between pre-F and post-F are blocked. In some embodiments, two or more epitopes of antigenic site I of RSV F are blocked. In some embodiments, one or more, or all, epitopes that topologically overlap with the blocked epitope are also blocked, optionally wherein the blocked epitope is an epitope of antigenic site I of RSV F.

In some embodiments, the RSV F polypeptide comprises an asparagine substitution at one or more positions corresponding to position 328, 348, or 507 of SEQ ID NO: 1 (i.e., E328N, S348N, or R507N). In some embodiments, the RSV F polypeptide comprises an asparagine substitution at two or more positions corresponding to position 328, 348, or 507 of SEQ ID NO: 1 (i.e., E328N, S348N, or R507N). In some embodiments, the RSV F polypeptide comprises an asparagine substitution at positions 328, 348, and 507 of SEQ ID NO: 1 (i.e., E328N, S348N, and R507N).

As shown previously, it has been found that such asparagines can function as glycosylation sites (see, WO2019/195291, incorporated herein by reference). Furthermore, without wishing to be bound by any particular theory, glycans at these sites may inhibit development of antibodies to nearby epitopes, which include epitopes common to pre- and post-fusion RSV F protein, when the RNA (e.g., mRNA) that encodes for the antigenic RSV F polypeptide is administered to a subject. In some embodiments, glycosylation of the asparagine corresponding to position 328, 348, or 507 of SEQ ID NO: 1 blocks at least one epitope shared between pre-fusion RSV F and post-fusion RSV F, such as an epitope of antigenic site 1. Inhibiting the development of antibodies to epitopes common to pre- and post-fusion RSV F protein can be beneficial because it can direct antibody development against epitopes specific to pre-fusion RSV F protein, such as the site Ø epitope, which may have more effective neutralizing activity than antibodies to other RSV F epitopes. The site Ø epitope involves amino acid residues 62-69 and 196-209 of SEQ ID NO: 1. Accordingly, in some embodiments, the RSV F polypeptide comprises amino acid residues 62-69 and 196-209 of SEQ ID NO: 1.

The RSV F polypeptides described herein may have deletions or substitutions of different length relative to wild type RSV F. For example, in the RSV F polypeptide of SEQ ID NO: 1, positions 98-144 of the wild-type sequence (SEQ ID NO: 1) are replaced with GSGNVGL (SEQ ID NO: 15), resulting in a net removal of 40 amino acids, such that positions 328, 348, or 507 of SEQ ID NO: 1 correspond to positions 288, 308, and 467 of SEQ ID NO: 3. In the alternative, in the RSV F polypeptide of SEQ ID NO: 3, positions 98-146 of the wild-type sequence (SEQ ID NO: 1) are replaced with GSGNVGLGG (SEQ ID NO: 16, positions 98-106 of SEQ ID NO: 3), resulting in a net removal of 40 amino acids, such that positions 328, 348, or 507 of SEQ ID NO: 1 correspond to positions 290, 310, and 469 of SEQ ID NO: 3.

In general, positions in constructs described herein can be mapped onto the wild-type sequence of SEQ ID NO: 1 by pairwise alignment, e.g., using the Needleman-Wunsch algorithm with standard parameters (EBLOSUM62 matrix, Gap penalty 10, gap extension penalty 0.5). See also the discussion of structural alignment provided herein as an alternative approach for identifying corresponding positions.

In some embodiments, the RSV F polypeptide comprises mutations that add glycans to block epitopes on the pre-fusion antigen that are structurally similar to those on the surface of the post-fusion RSV F. In some embodiments, glycans are added to specifically block epitopes that may be present in the post-fusion conformation of RSV F. In some embodiments, glycans are added that block epitopes that may be present in the post-fusion conformation of RSV F but do not affect one or more epitopes present on the pre-fusion conformation of RSV F, such as the site Ø epitope.

In some embodiments, the RSV F polypeptide comprises a sequence having at least 85%, 90%, 95%, 97%, 98%, 99%, or 99.5% identity to an amino acid sequence set forth in SEQ ID NO: 2.

In some embodiments, the RSV F polypeptide comprises a sequence having at least 85%, 90%, 95%, 97%, 98%, 99%, or 99.5% identity to an amino acid sequence set forth in SEQ ID NO: 3.

In some embodiments, the RSV F polypeptide comprises the DS-CAV1 amino acid substitutions (as described, for example, in McLellan et al., Science, 342(6158): 592-598, 2013) in which further modifications are made including at least one, two, or three of the asparagines described above. The CAV1 mutations are S190F and V207L relative to SEQ ID NO: 1. The DS mutations are S155C and S290C relative to SEQ ID NO: 1.

In some embodiments, an amino acid substitution or pair of amino acid substitutions are inter-protomer stabilizing substitution(s). Exemplary substitutions that can be inter-protomer stabilizing are V207L; N228F; 1217V and E218F; I221L and E222M; or Q224A and Q225L, using the position numbering of SEQ ID NO: 1.

In some embodiments, an amino acid substitution or pair of amino acid substitutions are intra-protomer stabilizing. Exemplary substitutions that can be intra-protomer stabilizing are V2201; and A74L and Q81L, using the position numbering of SEQ ID NO: 1.

In some embodiments, an amino acid substitution is helix stabilizing, i.e., predicted to stabilize the helical domain of RSV F. Stabilization of the helical domain can contribute to the stability of the site Ø epitope and of the pre-fusion conformation of RSV F generally. Exemplary substitutions that can be helix stabilizing are N216P or 1217P, using the position numbering of SEQ ID NO: 1. Position 217 in SEQ ID NO: 1 corresponds to position 177 in SEQ ID NO: 3.

In some embodiments, an amino acid substitution is helix capping. In some embodiments, an amino acid substitution is helix PRO capping. Helix capping is based on the biophysical observation that, while a proline residue mutation placed in an alpha helix may disrupt the helix formation, a proline at the N-terminus of a helical region may help induce helical formation by stabilizing the PHI/PSI bond angles. Exemplary substitutions that can be helix capping are N216P or 1217P, using the position numbering of SEQ ID NO: 1.

In some embodiments, an amino acid substitution replaces a disulfide mutation of DS-CAV1. In some embodiments, the engineered disulfide of DS-CAV1 is reverted to wild-type (C69S and/or C212S mutations of DS-CAV1 using the position numbering of SEQ ID NO: 1). In some embodiments, one or more C residue of DS-CAV1 is replaced with a S residue to eliminate a disulfide bond. In some embodiments, C69S or C212S substitution using the position numbering of SEQ ID NO: 1 eliminates a disulfide bond. In some embodiments, an RSV F polypeptide comprises both C69S and C212S using the position numbering of SEQ ID NO: 1. In some embodiments, replacing such cysteines and thereby eliminating a disulfide bond blocks reduction (i.e., acceptance of electrons from a reducing agent) of the RSV F polypeptide. In some embodiments, an 1217P substitution using the position numbering of SEQ ID NO: 1 is comprised in an antigen instead of substitution at C69 and/or C212.

In some embodiments, an amino acid substitution prevents proteolysis by trypsin or trypsin-like proteases. In some embodiments, the amino acid substitution that prevents such proteolysis is in the heptad repeat region B (HRB) region of RSV F.

Appearance of fragments consistent with proteolysis of an RSV F polypeptide that comprised a wild-type HRB region suggested a lysine or arginine in this region was being targeted for proteolysis. An amino acid substitution to remove a K or R residue may be termed a knockout (KO). In some embodiments, a K or R is substituted for L or Q. In some embodiments, a K is substituted for L or Q. In some embodiments, the RSV F polypeptide comprises K498L and/or K508Q, using the position numbering of SEQ ID NO: 1. The corresponding positions in SEQ ID NO: 3 are 458 and 468, respectively. In some embodiments, the RSV F polypeptide comprises both K498L and K508Q.

In some embodiments, an amino acid substitution adds glycans. In some embodiments, an amino acid substitution increases glycosylation by adding glycans to RSV F polypeptides. Substitutions to add glycans may also be referred to as engineered glycosylation, as compared to native glycosylation (without additional glycans).

In some embodiments, the amino acid substitution to add glycans is substitution with an N. In some embodiments, amino acid substitution with an N allows N-linked glycosylation. In some embodiments, substitution with an N is accompanied by substitution with a T or S at the second amino acid position C-terminal to the N, which forms an NxT/S glycosylation motif. In some embodiments, the N is surface-exposed.

Each of the above recited substitutions and mutations in the RSV F polypeptide are described in more detail in WO2019/195291, which is incorporated herein by reference.

In one aspect, the disclosure provides, a respiratory syncytial virus (RSV) vaccine, comprising a messenger RNA (mRNA) comprising an open reading frame (ORF) encoding an RSV F protein antigen, wherein the RSV F protein antigen comprises one or more of the following substitutions relative to an amino acid sequence set forth in SEQ ID NO: 1:
1) amino acid positions 98-146 of SEQ ID NO: 1 are replaced with an amino acid sequence of GSGNVGLGG (SEQ ID NO: 16);
2) amino acid substitutions 5190F and V207L;
3) amino acid substitution 1217P;
4) amino acid substitutions E328N, S348N, and R507N;
5) amino acid substitution L373R;
6) amino acid substitution K498L; and
7) amino acid substitution K508Q.

In another aspect, the disclosure provides an RSV vaccine comprising an mRNA comprising an ORF encoding an RSV F protein antigen, wherein the RSV F protein antigen comprises each of the following substitutions relative to an amino acid sequence set forth in SEQ ID NO: 1:
1) amino acid positions 98-146 of SEQ ID NO: 1 are replaced with an amino acid sequence of GSGNVGLGG (SEQ ID NO: 16);
2) amino acid substitutions 5190F and V207L;
3) amino acid substitution 1217P;
4) amino acid substitutions E328N, S348N, and R507N;
5) amino acid substitution L373R;
6) amino acid substitution K498L; and
7) amino acid substitution K508Q.

In certain embodiments, the RSV F protein antigen comprises a transmembrane domain and cytoplasmic tail amino acid sequence of IMITTIIIVIIVILLSLIAVGLLLYCKARSTPVTLSKDQLSGINNIAFSN (SEQ ID NO: 17).

In some embodiments, the mRNA comprises a nucleic acid sequence with at least 80%, at least 85%, at least 90%, at least 91%, at least 92%, at least 93%, at least 94%, at least 95%, at least 96%, at least 97%, at least 98%, at least 99%, or 100% identity to a nucleic acid sequence set forth in any one of SEQ ID NOs: 4-6.

In some embodiments, the mRNA comprises a nucleic acid sequence with at least 80%, at least 85%, at least 90%, at least 91%, at least 92%, at least 93%, at least 94%, at least 95%, at least 96%, at least 97%, at least 98%, at least 99%, or 100% identity to a nucleic acid sequence set forth in any one of SEQ ID NOs: 12-14.

### IV. Lipid Nanoparticle (LNP)

The LNPs of the disclosure can comprise four categories of lipids: (i) an ionizable lipid (e.g., cationic lipid); (ii) a PEGylated lipid; (iii) a cholesterol-based lipid (e.g., cholesterol), and (iv) a helper lipid.

A. Cationic Lipid

An ionizable lipid facilitates mRNA encapsulation and may be a cationic lipid. A cationic lipid affords a positively charged environment at low pH to facilitate efficient encapsulation of the negatively charged mRNA drug substance. Exemplary cationic lipids are shown below in Table 1.

TABLE 1
Ionizable Lipids

| Name | Structure |
|---|---|
| OF-02 (ML7) | 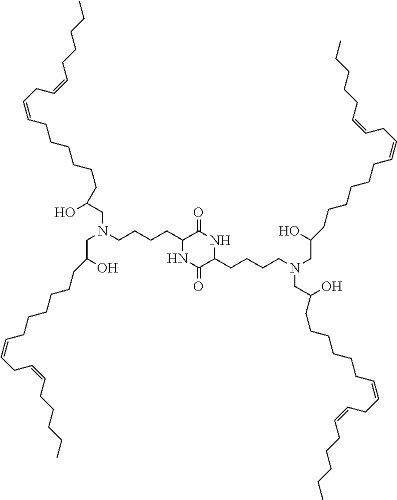 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC] |
| | OF-02 |
| cKK-E10 | 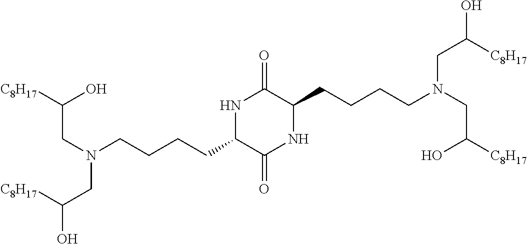 |
| GL-HEPES-E3- E10-DS-3-E18- 1 ((2-(4-(2-((3- (Bis((Z)-2- hydroxyoctadec- 9-en-1- yl)amino)propyl) disulfaneyl)ethyl) piperazin-1- yl)ethyl 4-(bis(2- hydroxydecyl) amino)butanoate) | 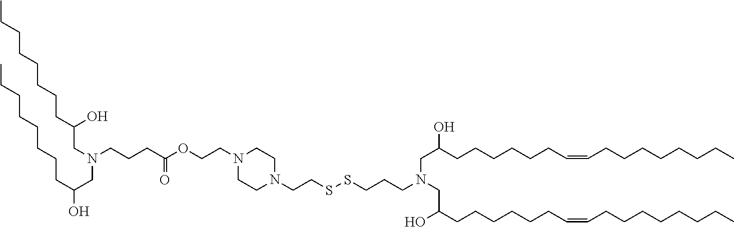 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC] |
| GL-HEPES-E3- E12-DS-4-E10 (2-(4-(2-((3- (bis(2- hydroxydecyl) amino)butyl) disulfaneyl)ethyl) piperazin-1-yl)ethyl 4-(bis(2- hydroxydodecyl) amino)butanoate) | 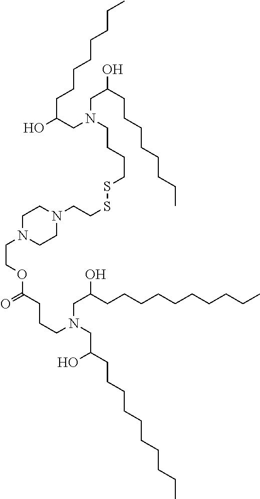 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC] |
| GL-HEPES-E3- E12-DS-3-E14 (2-(4-(2-((3- (Bis(2- hydroxytetradecyl) amino)propyl) disulfaneyl)ethyl) piperazin-1- yl)ethyl 4-(bis(2- hydroxydodecyl) amino)butanoate) | 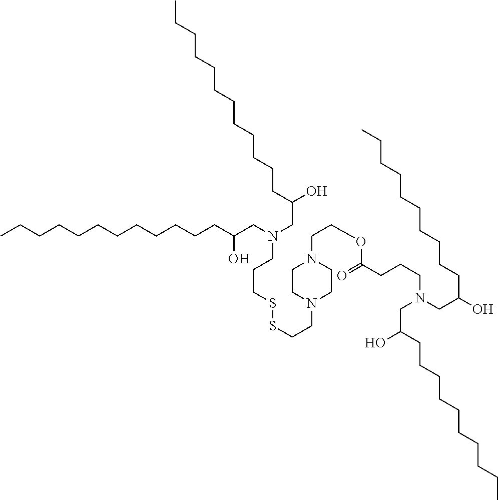 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC] |
| MC3 | 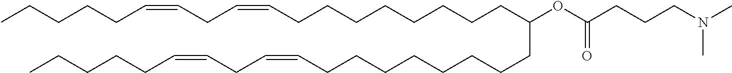 [SMILES: CCCCC/C=C\C/C=C\CCCCCCCCC(CCCCCCCC/C=C\C/C=C\CCCCC)OC(=O)CCCN(C)C] |
| SM-102 (9-heptadecanyl 8-{(2- hydroxyethyl)[6- oxo-6- (undecyloxy) hexyl]amino} octanoate) | 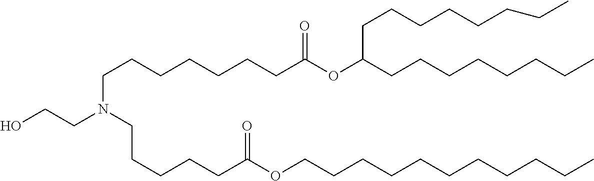 [SMILES: CCCCCCCCCCCOC(=O)CCCCCN(CCO)CCCCCCCC(=O)OC(CCCCCCCC)CCCCCCCC] |
| ALC-0315 [(4- hydroxybutyl) azanediyl]di (hexane-6,1-diyl) | 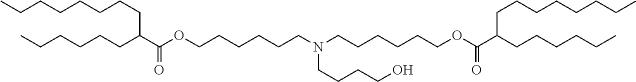 [SMILES: CCCCCCCCC(CCCCCC)C(=O)OCCCCCCN(CCCCO)CCCCCCOC(=O)C(CCCCCC)CCCCCCCC] |
| bis(2- | |
| hexyldecanoate) | |

The cationic lipid may be selected from the group comprising [ckkE10]/[OF-02], [(6Z,9Z,28Z,31Z)-heptatriaconta-6,9,28,31-tetraen-19-yl] 4-(dimethylamino)butanoate (D-Lin-MC3-DMA); 2,2-dilinoleyl-4-dimethylaminoethyl[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl] (Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl] N-[2-(dimethylamino)ethyl]carbamate (DC-Chol); tetrakis(8-methylnonyl) 3,3′,3″,3′″-(((methylazanediyl) bis(propane-3,1 diyl))bis (azanetriyl))tetrapropionate (306Oi10); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-O16B); 1,1′-((2-(4-(2′-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4,1-diyl))bis(azanetriyl))tetrakis(ethane-2,1-diyl) (9Z,9′Z,9″Z,9″Z,12Z,12′Z,12″Z,12′″Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino)octanoate (Lipid 5); and combinations thereof.

In certain embodiments, the cationic lipid is biodegradable.

In various embodiments, the cationic lipid is not biodegradable.

In some embodiments, the cationic lipid is cleavable.

In certain embodiments, the cationic lipid is not cleavable.

Cationic lipids are described in further detail in Dong et al. (PNAS. 111(11):3955-60. 2014); Fenton et al. (Adv. Mater. 28:2939. 2016); U.S. Pat. Nos. 9,512,073; and 10,201,618, each of which is incorporated herein by reference.

B. PEGylated Lipid

The PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al. FEBS Letters 268(1):235-7. 1990). These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or C₁₈) length, such as a derivatized ceramide (e.g., N-octanoyl-sphingosine-1-[succinyl(methoxypolyethylene glycol)] (C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

C. Cholesterol-Based Lipid

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol),1,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO2011/068810), sitosterol (22,23-dihydrostigmasterol), β-sitosterol, sitostanol, fucosterol, stigmasterol (stigmasta-5,22-dien-3-ol), ergosterol; desmosterol (3β-hydroxy-5,24-cholestadiene); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-3β-ol); lathosterol (5α-cholest-7-en-3β-ol); diosgenin ((3β,25R)-spirost-5-en-3β-ol); campesterol (campest-5-en-3β-ol); campestanol (5α-campestan-3b-ol); 24-methylene cholesterol (5,24(28)-cholestadien-24-methylen-3β-ol); cholesteryl margarate (cholest-5-en-3β-yl heptadecanoate); cholesteryl oleate; cholesteryl stearate and other modified forms of cholesterol. In some embodiments, the cholesterol-based lipid used in the LNPs is cholesterol.

D. Helper Lipid

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-I-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, I-stearoyl-2-oleoyl-phosphatidyethanolamine (SOPE), or a combination thereof. In certain embodiments, the helper lipid is DOPE. In certain embodiments, the helper lipid is DSPC.

In various embodiments, the present LNPs comprise (i) a cationic lipid selected from OF-02, cKK-E10, GL-HEPES-E3-E10-DS-3-E18-1, GL-HEPES-E3-E12-DS-4-E10, or GL-HEPES-E3-E12-DS-3-E14; (ii) DMG-PEG2000; (iii) cholesterol; and (iv) DOPE.

E. Molar Ratios of the Lipid Components

The molar ratios of the above components are important for the LNPs' effectiveness in delivering mRNA. The molar ratio of the cationic lipid, the PEGylated lipid, the cholesterol-based lipid, and the helper lipid is A:B:C:D, where A+B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12%, such as 10%). In some embodiments, the (PEGylated lipid+cholesterol) components have the same molar amount as the helper lipid. In some embodiments, the LNPs contain a molar ratio of the cationic lipid to the helper lipid that is more than 1.

In certain embodiments, the LNP of the disclosure comprises:
a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%);
a polyethylene glycol (PEG) conjugated (PEGylated) lipid at a molar ratio of 0.25% to 2.75% or 1.00% to 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%);
a cholesterol-based lipid at a molar ratio of 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, or 50%); and
a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%),
wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the PEGylated lipid is dimyristoyl-PEG2000 (DMG-PEG2000).

In various embodiments, the cholesterol-based lipid is cholesterol.

In some embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 ata molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-3-E14 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%. This LNP formulation is designated “Lipid A” herein.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid B” herein.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%. This LNP formulation is designated “Lipid C” herein.

In certain embodiments, the LNP comprises: GL-HEPES-E3-E12-DS-4-E10 (at a molar ratio of 40%; DMG-PEG2000 at a molar ratio of 1.5%; cholesterol at a molar ratio of 28.5%; and DOPE at a molar ratio of 30%. This LNP formulation is designated “Lipid D” herein.

In certain embodiments, the LNP comprises: GL-HEPES-E3-E12-DS-3-E14 at a molar ratio of 40%; DMG-PEG2000 at a molar ratio of 1.5%; cholesterol at a molar ratio of 28.5%; and DOPE at a molar ratio of 30%. This LNP formulation is designated “Lipid E” herein.

In certain embodiments, the LNP comprises: 9-heptadecanyl 8-{(2-hydroxyethyl)[6-oxo-6-(undecyloxy)hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl)azanediyl]di(hexane-6,1-diyl) bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl)azanediyl]di(hexane-6,1-diyl) bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

F. Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients are parabens, thimerosal, thiomersal, chlorobutanol, benzalkonium chloride, and chelators (e.g., EDTA).

The LNP compositions of the present disclosure can be provided as a frozen liquid form or a lyophilized form. A variety of cryoprotectants may be used, including, without limitation, sucrose, trehalose, glucose, mannitol, mannose, dextrose, and the like. The cryoprotectant may constitute 5-30% (w/v) of the LNP composition. In some embodiments, the LNP composition comprise trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution—thawed if previously frozen, or if previously lyophilized, reconstituted in an aqueous buffered solution at bedside. The buffered solution can be isotonic and suitable, e.g., for intramuscular or intradermal injection. In some embodiments, the buffered solution is a phosphate-buffered saline (PBS).

### V. Processes for Making LNP Vaccines

The present LNPs can be prepared by various techniques. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication, or extrusion of the multilamellar vesicles. In addition, unilamellar vesicles can be formed by detergent removal techniques.

Various methods are described in US 2011/0244026, US 2016/0038432, US 2018/0153822, US 2018/0125989, and US 2021/0046192 and can be used for making LNP vaccines. One exemplary process entails encapsulating mRNA by mixing it with a mixture of lipids, without first pre-forming the lipids into lipid nanoparticles, as described in US 2016/0038432. Another exemplary process entails encapsulating mRNA by mixing pre-formed LNPs with mRNA, as described in US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA, and the mixed solution comprising the LNP-encapsulated mRNA. In some embodiments, the process includes the step of heating one or both of the mRNA solution and the pre-formed LNP solution prior to the mixing step. In some embodiments, the process includes heating one or more of the solutions comprising the pre-formed LNPs, the solution comprising the mRNA, and the solution comprising the LNP-encapsulated mRNA during the mixing step. In some embodiments, the process includes the step of heating the LNP-encapsulated mRNA after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present disclosure. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include, but are not limited to, gear pumps, peristaltic pumps, and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging from about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging from about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods are available for sizing of a population of lipid nanoparticles. In various embodiments, methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80% or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In certain embodiments, the LNP has an average diameter of 30-200 nm.

In various embodiments, the LNP has an average diameter of 80-150 nm.

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, or 99% of the LNPs in the present composition have a size ranging from about 40-90 nm (e.g., about 45-85 nm, about 50-80 nm, about 55-75 nm, or about 60-70 nm) or about 50-70 nm (e.g., about 55-65 nm) are suitable for pulmonary delivery via nebulization.

In some embodiments, the dispersity, or measure of heterogeneity in size of molecules (PDI), of LNPs in a pharmaceutical composition provided by the present disclosure is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of 50% to 99%; or greater than about 60%, 65%, 70%, 75%, 80%, 85%, 90%, 92%, 95%, 98%, or 99%. Typically, lipid nanoparticles for use herein have an encapsulation efficiency of at least 90% (e.g., at least 91%, 92%, 93%, 94%, or 95%).

In some embodiments, an LNP has a N/P ratio of 1 to 10. In some embodiments, a lipid nanoparticle has a N/P ratio above 1, about 1, about 2, about 3, about 4, about 5, about 6, about 7, or about 8. In certain embodiments, a typical LNP herein has an N/P ratio of 4.

In some embodiments, a pharmaceutical composition according to the present disclosure contains at least about 0.5 μg, 1 μg, 5 μg, 10 μg, 100 μg, 500 μg, or 1000 μg of encapsulated mRNA. In some embodiments, a pharmaceutical composition contains about 0.1 μg to 1000 μg, at least about 0.5 μg, at least about 0.8 μg, at least about 1 μg, at least about 5 μg, at least about 8 μg, at least about 10 μg, at least about 50 μg, at least about 100 μg, at least about 500 μg, or at least about 1000 μg of encapsulated mRNA.

In some embodiments, mRNA can be made by chemical synthesis or by in vitro transcription (IVT) of a DNA template. An exemplary process for making and purifying mRNA is described in Example 1. In this process, an IVT process, a cDNA template is used to produce an mRNA transcript and the DNA template is degraded by a DNase. The transcript is purified by depth filtration and tangential flow filtration (TFF). The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In particular embodiments, the aqueous buffer has 1 mM citrate, 150 mM NaCl, pH 4.5.

An exemplary, nonlimiting process for making an mRNA-LNP composition involves mixing a buffered mRNA solution with a solution of lipids in ethanol in a controlled homogeneous manner, where the ratio of lipids:mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

### VI. Packaging and Use of the mRNA-LNP RSV Vaccine

The mRNA-LNP vaccines can be formulated or packaged for parenteral (e.g., intramuscular, intradermal, or subcutaneous) administration or nasopharyngeal (e.g., intranasal) administration. In various embodiments, the mRNA-LNP vaccines may be formulated or packaged for pulmonary administration. In various embodiments, the mRNA-LNP vaccines may be formulated or packaged for intravenous administration. The vaccine compositions may be in the form of an extemporaneous formulation, where the LNP composition is lyophilized and reconstituted with a physiological buffer (e.g., PBS) just before use. The vaccine compositions also may be shipped and provided in the form of an aqueous solution or a frozen aqueous solution and can be directly administered to subjects without reconstitution (after thawing, if previously frozen).

Accordingly, the present disclosure provides an article of manufacture, such as a kit, that provides the mRNA-LNP vaccine in a single container or provides the mRNA-LNP vaccine in one container (e.g., a first container) and a physiological buffer for reconstitution in another container (e.g., a second container). The container(s) may contain a single-use dosage or multi-use dosage. The container(s) may be pre-treated glass vials or ampules. The article of manufacture may include instructions for use as well.

In certain embodiments, the mRNA-LNP vaccine is provided for use in intramuscular (IM) injection. The vaccine can be injected into a subject at, e.g., his/her deltoid muscle in the upper arm. In some embodiments, the vaccine is provided in a pre-filled syringe or injector (e.g., single-chambered or multi-chambered). In some embodiments, the vaccine is provided for use in inhalation and is provided in a pre-filled pump, aerosolizer, or inhaler.

The mRNA-LNP vaccines can be administered to subjects in need thereof in a prophylactically effective amount, i.e., an amount that provides sufficient immune protection against a target pathogen for a sufficient amount of time (e.g., one year, two years, five years, ten years, or a lifetime). Sufficient immune protection may be, for example, prevention or alleviation of symptoms associated with infections by the pathogen. In some embodiments, multiple doses (e.g., two doses) of the vaccine are administered (e.g., injected) to subjects in need thereof to achieve the desired prophylactic effects. The doses (e.g., prime and booster doses) may be separated by an interval of at least, e.g., 2 weeks, 3 weeks, 4 weeks, one month, two months, three months, four months, five months, six months, one year, two years, five years, or ten years.

### VII. Vectors

In one aspect, disclosed herein are vectors comprising the mRNA compositions disclosed herein. The RNA sequences encoding a protein of interest (e.g., mRNA encoding an RSV F protein) can be cloned into a number of types of vectors. For example, the nucleic acids can be cloned into a vector including, but not limited to, a plasmid, a phagemid, a phage derivative, an animal virus, and a cosmid. Vectors of particular interest can include expression vectors, replication vectors, probe generation vectors, sequencing vectors, and vectors optimized for in vitro transcription.

In certain embodiments, the vector can be used to express mRNA in a host cell. In various embodiments, the vector can be used as a template for IVT. The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14,265-281.

In some embodiments, the vectors disclosed herein can comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein may comprise a polynucleotide sequence encoding a poly(A) sequence and/or a polyadenylation signal.

A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known.

Also disclosed herein are host cells (e.g., mammalian cells, e.g., human cells) comprising the vectors or RNA compositions disclosed herein.

Polynucleotides can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12(8):861-70, or the TransIT-RNA transfection Kit (Mirus, Madison, WI).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell a variety of assays may be performed.

### VIII. Self-Replicating RNA and Trans-Replicating RNA

Self-Replicating RNA:

In one aspect, disclosed herein are self-replicating RNAs encoding an RSV F protein.

Self-replicating RNA can be produced by using replication elements derived from, e.g., alphaviruses, and substituting the structural viral proteins with a nucleotide sequence encoding a protein of interest (e.g., RSV F protein). A self-replicating RNA is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen (i.e., an RSV F protein antigen), or may be transcribed to provide further transcripts with the same sense as the delivered RNA which are translated to provide in situ expression of the antigen. The overall result of this sequence of transcriptions is a large amplification in the number of the introduced replicon RNAs and so the encoded antigen becomes a major polypeptide product of the cells.

One suitable system for achieving self-replication in this manner is to use an alphavirus-based replicon. These replicons are positive stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the positive-strand delivered RNA. These negative (−)-stranded transcripts can themselves be transcribed to give further copies of the positive-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, see the following reference: WO2005/113782, incorporated herein by reference.

In one embodiment, each self-replicating RNA described herein encodes (i) an RNA-dependent RNA polymerase which can transcribe RNA from the self-replicating RNA molecule and (ii) a RSV F protein antigen. The polymerase can be an alphavirus replicase, e.g., comprising one or more of alphavirus proteins nsP1, nsP2, nsP3, and nsP4. Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins. Self-replicating RNA are described in further detail in WO2011005799, incorporated herein by reference.

Trans-Replicating RNA:

In one aspect, disclosed herein are trans-replicating RNAs encoding an RSV F protein.

Trans-replicating RNA possess similar elements as the self-replicating RNA described above. However, with trans replicating RNA, two separate RNA molecules are used. A first RNA molecule encodes for the RNA replicase described above (e.g., the alphavirus replicase) and a second RNA molecule encodes for the protein of interest (e.g., an RSV F protein antigen). The RNA replicase may replicate one or both of the first and second RNA molecule, thereby greatly increasing the copy number of RNA molecules encoding the protein of interest. Trans replicating RNA are described in further detail in WO2017162265, incorporated herein by reference.

### IX. Pharmaceutical Compositions

RNA purified according to this disclosure can be useful as a component in pharmaceutical compositions, for example, for use as a vaccine. These compositions will typically include RNA and a pharmaceutically acceptable carrier. A pharmaceutical composition of the present disclosure can also include one or more additional components such as small molecule immunopotentiators (e.g., TLR agonists). A pharmaceutical composition of the present disclosure can also include a delivery system for the RNA, such as a liposome, an oil-in-water emulsion, or a microparticle. In some embodiments, the pharmaceutical composition comprises a lipid nanoparticle (LNP). In certain embodiments, the composition comprises an antigen-encoding nucleic acid molecule encapsulated within a LNP.

### X. Methods of Vaccination

The RSV vaccine disclosed herein may be administered to a subject to induce an immune response directed against the RSV F protein, wherein an anti-antigen antibody titer in the subject is increased following vaccination relative to an anti-antigen antibody titer in a subject that is not vaccinated with the RSV vaccine disclosed herein, or relative to an alternative vaccine against RSV. An “anti-antigen antibody” is a serum antibody that binds specifically to the antigen.

In one aspect, the disclosure provides a method of eliciting an immune response to RSV or protecting a subject against RSV infection comprising administering the RSV vaccine described herein to a subject. The disclosure also provides an RSV vaccine described herein for use in eliciting an immune response to RSV or in protecting a subject against RSV infection. The disclosure also provides an RSV mRNA described herein for use in the manufacture of a vaccine for eliciting an immune response to RSV or for protecting a subject against RSV infection.

In certain embodiments, the subject has a higher serum concentration of neutralizing antibodies against RSV after administration of the RSV vaccine, relative to a subject that is administered an RSV vaccine comprising an mRNA ORF encoding an RSV F protein antigen of SEQ ID NO: 1.

In certain embodiments, the subject has a comparable serum concentration of neutralizing antibodies against RSV after administration of the RSV vaccine, relative to a subject that is administered an RSV protein vaccine that is co-administered with an adjuvant.

In certain embodiments, the RSV vaccine increases the serum concentration of antibodies with binding specificity to site Ø of the RSV F protein.

In certain embodiments, the subject has a lower serum concentration of antibodies with binding specificity to site I or site II of the RSV F protein after administration of the RSV vaccine, relative to a subject that is administered an RSV vaccine comprising an mRNA ORF encoding an RSV F protein antigen of SEQ ID NO: 2.

In certain embodiments, the RSV vaccine increases the serum concentration of neutralizing antibodies in a subject with pre-existing RSV immunity.

In order that this invention may be better understood, the following examples are set forth. These examples are for purposes of illustration only and are not to be construed as limiting the scope of the invention in any manner.

### EXAMPLES

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### Example 1: mRNA Encoding RSV F Protein

Three different RSV F proteins were selected for testing as an mRNA-based vaccine. The F protein designated FD1 corresponds to WT RSV F protein. The F protein designated FD2 corresponds to a soluble RSV F protein lacking the transmembrane domain and cytoplasmic tail and containing a C terminal fibritin trimerization domain (also known as T4 foldon). The F protein designated FD3 corresponds to a pre-fusion RSV F protein. The amino acid sequences for each of the RSV F proteins are recited below.

FD1:
(SEQ ID NO: 1)
MELLILKANAITTILTAVTFCFASGQNITEEFYQSTCSAVSKGYLSALR
TGWYTSVITIELSNIKKNKCNGTDAKVKLIKQELDKYKNAVTELQLLMQ
STQATNNRARRELPRFMNYTLNNAKKTNVTLSKKRKRRFLGFLLGVGSA
IASGVAVSKVLHLEGEVNKIKSALLSTNKAVVSLSNGVSVLTSKVLDLK
NYIDKQLLPIVNKQSCSISNIETVIEFQQKNNRLLEITREFSVNAGVTT
PVSTYMLTNSELLSLINDMPITNDQKKLMSNNVQIVRQQSYSIMSIIKE
EVLAYVVQLPLYGVIDTPCWKLHTSPLCTTNTKEGSNICLTRTDRGWYC
DNAGSVSFFPQAETCKVQSNRVFCDTMNSLTLPSEVNLCNVDIFNPKYD
CKIMTSKTDVSSSVITSLGAIVSCYGKTKCTASNKNRGIIKTFSNGCDY
VSNKGVDTVSVGNTLYYVNKQEGKSLYVKGEPIINFYDPLVFPSDEFDA
SISQVNEKINQSLAFIRKSDELLHNVNAGKSTTNIMITTIIIVIIVILL
SLIAVGLLLYCKARSTPVTLSKDQLSGINNIAFSN
FD2:
(SEQ ID NO: 2)
MELLILKANAITTILTAVTFCFASGQNITEEFYQSTCSAVSKGYLSALR
TGWYTSVITIELSNIKKNKCNGTDAKVKLIKQELDKYKNAVTELQLLMQ
STQATNNRARRELPRFMNYTLNNAKKTNVTLSKKRKRRFLGFLLGVGSA
IASGVAVSKVLHLEGEVNKIKSALLSTNKAVVSLSNGVSVLTSKVLDLK
NYIDKQLLPIVNKQSCSISNIETVIEFQQKNNRLLEITREFSVNAGVTT
PVSTYMLTNSELLSLINDMPITNDQKKLMSNNVQIVRQQSYSIMSIIKE
EVLAYVVQLPLYGVIDTPCWKLHTSPLCTTNTKEGSNICLTRTDRGWYC
DNAGSVSFFPQAETCKVQSNRVFCDTMNSLTLPSEVNLCNVDIFNPKYD
CKIMTSKTDVSSSVITSLGAIVSCYGKTKCTASNKNRGIIKTFSNGCDY
VSNKGVDTVSVGNTLYYVNKQEGKSLYVKGEPIINFYDPLVFPSDEFDA
SISQVNEKINQSLAFIRKSDELLSAIGGYIPEAPRDGQAYVRKDGEWLL
STFL
FD3:
(SEQ ID NO: 3)
MELLIKANAITTILTAVTFCFASGQNITEEFYQSTCSAVSKGYLSALRT
GWYTSVITIELSNIKENKCNGTDAKVKLIKQELDKYKNAVTELQLLMGS
GNVGLGGAIASGVAVSKVLHLEGEVNKIKSALLSTNKAVVSLSNGVSVL
TFKVLDLKNYIDKQLLPILNKQSCSISNPETVIEFQQKNNRLLEITREF
SVNAGVTTPVSTYMLTNSELLSLINDMPITNDQKKLMSNNVQIVRQQSY
SIMSIIKEEVLAYVVQLPLYGVIDTPCWKLHTSPLCTTNTKNGSNICLT
RTDRGWYCDNAGNVSFFPQAETCKVQSNRVFCDTMNSRTLPSEVNLCNV
DIFNPKYDCKIMTSKTDVSSSVITSLGAIVSCYGKTKCTASNKNRGIIK
TFSNGCDYVSNKGVDTVSVGNTLYYVNKQEGKSLYVKGEPIINFYDPLV
FPSDEFDASISQVNELINQSLAFINQSDELLHNVNAGKSTTNIMITTII
IVIIVILLSLIAVGLLLYCKARSTPVTLSKDQLSGINNIAFSN

The mRNA described herein comprise an open reading frame (ORF) encoding an RSV F protein antigen, at least one 5′ untranslated region (5′ UTR), at least one 3′ untranslated region (3′ UTR), and at least one polyadenylation (poly(A)) sequence. The mRNA further comprises a 5′ cap with the following structure:

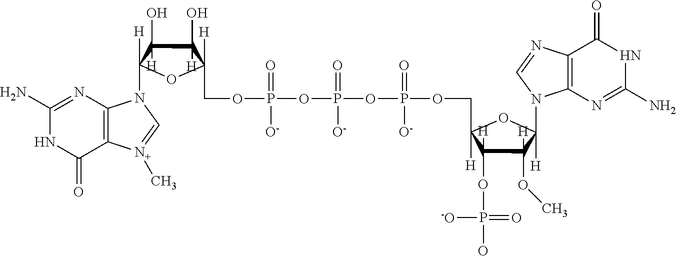

The nucleic acid sequences for each of the mRNA open reading frames (ORFs) encoding the RSV F proteins are recited below.

FD1 mRNA ORF:
(SEQ ID NO: 4)
AUGGAAUUGCUGAUCCUCAAAGCGAACGCAAUCACCACUAUCCUCACUGCGGUCACCUUCU
GCUUUGCGAGCGGACAGAACAUCACCGAAGAAUUCUACCAAUCUACUUGCUCCGCCGUGUC
CAAGGGUUACCUGUCCGCCCUGAGGACCGGAUGGUACACUUCCGUGAUUACCAUUGAGUU
GUCGAAUAUCAAGAAGAACAAGUGCAACGGAACCGAUGCUAAGGUCAAGCUGAUCAAGCAG
GAGCUGGACAAGUACAAGAAUGCUGUGACCGAGCUGCAGCUGCUGAUGCAGUCCACUCAAG
CCACCAACAAUCGCGCCCGGCGGGAACUCCCAAGGUUCAUGAACUACACCUUGAACAACGC
CAAGAAAACGAACGUGACCCUGUCCAAGAAGCGCAAGCGCAGAUUCCUUGGCUUCCUUCUG
GGCGUCGGUAGCGCCAUCGCCUCCGGCGUGGCCGUCAGCAAGGUCCUGCACCUCGAGGGA
GAAGUCAACAAGAUUAAGAGCGCCCUGCUGUCCACCAACAAGGCCGUGGUGUCGCUAUCAA
ACGGCGUCAGCGUACUGACCAGCAAAGUGCUGGAUCUCAAGAACUACAUUGAUAAGCAACU
CCUCCCUAUCGUGAAUAAGCAGAGCUGUUCGAUUUCCAACAUCGAGACUGUGAUUGAAUUC
CAGCAGAAGAACAACCGGCUGCUGGAAAUUACCAGAGAAUUCAGCGUGAAUGCCGGAGUCA
CUACCCCCGUGUCCACCUACAUGCUGACAAACUCCGAGCUGCUGAGCCUGAUCAACGAUAU
GCCGAUUACCAACGACCAGAAGAAGCUGAUGUCGAACAACGUGCAGAUCGUGCGCCAGCAG
UCCUACUCAAUCAUGUCGAUCAUCAAGGAAGAGGUCCUGGCCUACGUGGUGCAGCUUCCUC
UGUACGGCGUGAUUGACACUCCGUGUUGGAAACUGCACACUAGUCCCCUGUGCACUACUAA
CACCAAGGAGGGCAGCAAUAUCUGCCUGACUCGGACCGAUAGAGGCUGGUACUGUGAUAAC
GCCGGGUCCGUGUCCUUCUUCCCGCAAGCCGAGACUUGCAAAGUGCAGAGCAACCGGGUG
UUCUGUGACACUAUGAACUCACUGACCUUGCCGAGCGAAGUCAACCUUUGCAACGUGGACA
UCUUUAACCCUAAAUACGACUGCAAGAUCAUGACCUCCAAGACCGACGUGUCGAGCUCAGU
GAUUACUUCGCUGGGAGCCAUUGUGUCCUGCUACGGGAAAACCAAGUGCACGGCCUCAAAC
AAGAACCGGGGUAUCAUUAAGACCUUCUCCAACGGCUGCGACUAUGUGUCCAACAAGGGGG
UGGACACUGUGUCCGUGGGAAACACCUUGUAUUACGUGAACAAGCAGGAGGGAAAGUCCCU
CUACGUGAAGGGCGAACCCAUCAUCAAUUUCUACGACCCGCUCGUGUUCCCCUCCGAUGAA
UUCGACGCAUCCAUCUCACAAGUCAACGAAAAGAUUAACCAGUCCCUGGCUUUCAUUCGCA
AGUCCGACGAACUGCUCCAUAACGUCAACGCUGGAAAGUCCACCACCAACAUCAUGAUCAC
CACGAUCAUUAUUGUGAUCAUCGUCAUCCUGCUGUCACUGAUAGCAGUGGGACUGCUCCUC
UACUGCAAAGCGCGGUCGACCCCAGUGACACUCUCGAAGGACCAGCUGUCCGGGAUCAACA
ACAUCGCGUUUUCGAACUGA
FD2 mRNA ORF:
(SEQ ID NO: 5)
AUGGAACUCCUGAUCCUGAAGGCCAAUGCUAUCACUACCAUCCUGACUGCCGUCACCUUCU
GCUUCGCCUCCGGACAAAAUAUCACUGAAGAAUUUUACCAAAGCACCUGUAGCGCGGUGUC
CAAGGGAUACCUGAGCGCUCUGAGGACCGGAUGGUACACCAGCGUGAUUACCAUCGAGCU
GAGUAACAUCAAGAAGAACAAGUGCAACGGGACCGAUGCUAAGGUCAAGUUGAUCAAACAA
GAGCUCGACAAGUACAAGAACGCCGUGACUGAGCUGCAGCUGCUGAUGCAGUCAACUCAGG
CCACCAACAACCGGGCCAGACGGGAACUGCCGAGAUUCAUGAACUACACCCUGAACAACGC
CAAAAAGACCAACGUGACCCUGUCCAAGAAGAGAAAGCGCCGGUUCCUGGGUUUCCUGCUU
GGCGUGGGAUCAGCAAUCGCGUCCGGAGUGGCAGUGUCCAAGGUCUUGCACCUCGAGGGC
GAAGUGAACAAGAUCAAGUCCGCGCUUCUGUCGACCAACAAGGCCGUCGUUUCCCUGUCGA
ACGGAGUGUCCGUGCUCACGAGCAAAGUGCUCGACCUGAAGAACUACAUCGACAAACAGCU
GCUGCCCAUCGUCAACAAGCAGAGCUGCAGCAUCUCAAACAUUGAAACCGUGAUCGAGUUC
CAGCAGAAGAACAACCGCCUGCUCGAGAUUACCAGAGAGUUUUCCGUGAACGCCGGCGUGA
CCACCCCGGUGUCGACCUACAUGCUCACAAAUUCGGAACUUCUCUCCCUGAUUAAUGACAU
GCCCAUUACUAACGAUCAGAAAAAGCUGAUGUCGAACAAUGUGCAGAUUGUGCGCCAGCAG
UCCUACUCCAUCAUGUCCAUCAUUAAGGAAGAGGUCCUGGCCUACGUGGUGCAGUUGCCG
CUGUACGGUGUCAUCGAUACCCCCUGCUGGAAGCUCCAUACUUCGCCCCUGUGUACUACCA
ACACCAAGGAAGGCUCCAACAUCUGCCUGACCCGGACGGAUCGCGGCUGGUACUGUGACAA
UGCCGGAUCCGUGUCGUUCUUCCCGCAAGCGGAGACUUGCAAAGUGCAGUCCAACCGGGU
GUUCUGUGACACUAUGAACUCCCUGACUCUGCCGUCCGAAGUCAACCUCUGCAACGUGGAC
AUUUUCAAUCCAAAAUACGACUGCAAGAUAAUGACCUCCAAGACUGACGUGUCAUCGUCCG
UGAUCACAUCUCUGGGAGCCAUUGUCUCCUGCUACGGAAAGACUAAGUGCACCGCGUCGAA
CAAGAACAGGGGCAUUAUCAAGACCUUCAGCAACGGUUGCGACUAUGUGUCCAACAAGGGC
GUGGAUACCGUGUCCGUGGGCAACACCUUGUACUACGUGAACAAGCAGGAGGGGAAGUCC
CUUUAUGUGAAGGGGGAGCCAAUCAUUAACUUUUACGACCCCCUGGUGUUCCCUAGCGACG
AGUUCGACGCCUCAAUCUCUCAAGUCAACGAAAAGAUCAACCAGAGCCUCGCCUUCAUCCG
CAAGUCCGAUGAACUGCUGUCAGCCAUUGGGGGUUACAUCCCUGAGGCCCCUCGGGACGG
ACAGGCAUACGUCCGCAAGGACGGCGAAUGGGUGCUGCUUAGCACCUUCCUCUAA
FD3 mRNA ORF:
(SEQ ID NO: 6)
AUGGAACUGCUGAUCCUCAAAGCCAACGCAAUCACCACCAUUCUCACCGCUGUGACCUUCU
GCUUCGCAUCGGGGCAGAACAUCACUGAAGAGUUUUACCAGAGCACUUGCAGCGCGGUGU
CAAAGGGUUACCUUUCCGCACUGCGGACCGGAUGGUACACUUCCGUGAUCACCAUUGAGCU
CAGCAACAUCAAGGAAAACAAGUGCAAUGGCACCGACGCCAAGGUCAAGCUGAUCAAACAAG
AACUGGACAAGUACAAGAACGCCGUGACAGAAUUGCAGCUCCUGAUGGGAUCCGGAAACGU
CGGUCUGGGCGGAGCCAUCGCGAGUGGAGUGGCUGUGUCCAAGGUCUUGCACCUCGAGG
GAGAAGUGAACAAGAUCAAGUCCGCGCUGCUGUCAACGAACAAGGCCGUGGUGUCCCUGUC
UAACGGCGUCAGCGUGCUGACGUUCAAGGUCCUGGACCUGAAGAAUUACAUUGACAAGCAG
CUGCUGCCCAUCCUCAACAAGCAAUCCUGCUCCAUCUCCAACCCCGAAACCGUGAUCGAGU
UCCAGCAGAAGAACAACCGCCUGCUGGAAAUUACUCGCGAGUUCUCUGUGAAUGCCGGCGU
GACCACCCCUGUGUCCACCUACAUGCUGACCAACUCCGAGCUUCUCUCCCUUAUCAAUGAC
AUGCCUAUCACGAACGACCAGAAGAAGCUGAUGUCGAACAACGUGCAGAUUGUGCGGCAGC
AGUCAUACAGCAUCAUGUCGAUCAUCAAGGAAGAAGUGCUGGCGUACGUGGUGCAACUCCC
GCUGUACGGCGUCAUCGAUACCCCGUGCUGGAAGCUGCACACCUCGCCUUUGUGUACCAC
CAACACCAAGAACGGAUCCAACAUCUGCUUAACCCGGACUGAUCGGGGUUGGUACUGCGAC
AACGCCGGGAAUGUUUCGUUCUUCCCACAAGCCGAGACUUGUAAAGUGCAGUCAAACAGAG
UGUUCUGUGACACCAUGAACUCGAGAACCCUGCCCAGCGAAGUGAACCUGUGUAACGUCGA
CAUCUUUAACCCAAAAUACGAUUGCAAGAUUAUGACCAGCAAAACCGACGUGUCCUCCUCCG
UGAUAACAAGCCUGGGGGCGAUUGUGUCAUGCUACGGAAAGACUAAGUGCACCGCCUCGAA
CAAGAACCGCGGCAUCAUUAAGACUUUCUCGAAUGGUUGCGACUAUGUGUCCAACAAGGGC
GUGGAUACUGUGUCAGUCGGGAAUACUCUUUACUACGUGAACAAGCAGGAGGGGAAAAGCC
UCUACGUGAAGGGAGAGCCUAUUAUCAACUUUUACGAUCCGCUGGUGUUCCCGUCCGACGA
AUUCGACGCCAGCAUCAGCCAAGUCAACGAGCUGAUUAACCAGUCCCUCGCCUUCAUCAAC
CAAUCCGACGAGCUCCUGCAUAACGUGAACGCCGGAAAGUCCACCACCAACAUCAUGAUCA
CUACUAUUAUCAUCGUGAUCAUCGUCAUCCUGCUGAGCCUGAUUGCUGUGGGCCUGUUGC
UGUAUUGCAAAGCCAGGUCCACCCCGGUCACCCUGUCGAAGGAUCAGCUGUCCGGAAUCAA
CAACAUUGCCUUCUCCAACUAA

The nucleic acid sequences for each of the DNA templates encoding the RSV F proteins are recited below.

FD1 DNA:
(SEQ ID NO: 7)
ATGGAATTGCTGATCCTCAAAGCGAACGCAATCACCACTATCCTCACTGCGGTCACCTTCTGCT
TTGCGAGCGGACAGAACATCACCGAAGAATTCTACCAATCTACTTGCTCCGCCGTGTCCAAGG
GTTACCTGTCCGCCCTGAGGACCGGATGGTACACTTCCGTGATTACCATTGAGTTGTCGAATA
TCAAGAAGAACAAGTGCAACGGAACCGATGCTAAGGTCAAGCTGATCAAGCAGGAGCTGGAC
AAGTACAAGAATGCTGTGACCGAGCTGCAGCTGCTGATGCAGTCCACTCAAGCCACCAACAAT
CGCGCCCGGCGGGAACTCCCAAGGTTCATGAACTACACCTTGAACAACGCCAAGAAAACGAA
CGTGACCCTGTCCAAGAAGCGCAAGCGCAGATTCCTTGGCTTCCTTCTGGGCGTCGGTAGCG
CCATCGCCTCCGGCGTGGCCGTCAGCAAGGTCCTGCACCTCGAGGGAGAAGTCAACAAGATT
AAGAGCGCCCTGCTGTCCACCAACAAGGCCGTGGTGTCGCTATCAAACGGCGTCAGCGTACT
GACCAGCAAAGTGCTGGATCTCAAGAACTACATTGATAAGCAACTCCTCCCTATCGTGAATAAG
CAGAGCTGTTCGATTTCCAACATCGAGACTGTGATTGAATTCCAGCAGAAGAACAACCGGCTG
CTGGAAATTACCAGAGAATTCAGCGTGAATGCCGGAGTCACTACCCCCGTGTCCACCTACATG
CTGACAAACTCCGAGCTGCTGAGCCTGATCAACGATATGCCGATTACCAACGACCAGAAGAAG
CTGATGTCGAACAACGTGCAGATCGTGCGCCAGCAGTCCTACTCAATCATGTCGATCATCAAG
GAAGAGGTCCTGGCCTACGTGGTGCAGCTTCCTCTGTACGGCGTGATTGACACTCCGTGTTG
GAAACTGCACACTAGTCCCCTGTGCACTACTAACACCAAGGAGGGCAGCAATATCTGCCTGAC
TCGGACCGATAGAGGCTGGTACTGTGATAACGCCGGGTCCGTGTCCTTCTTCCCGCAAGCCG
AGACTTGCAAAGTGCAGAGCAACCGGGTGTTCTGTGACACTATGAACTCACTGACCTTGCCGA
GCGAAGTCAACCTTTGCAACGTGGACATCTTTAACCCTAAATACGACTGCAAGATCATGACCTC
CAAGACCGACGTGTCGAGCTCAGTGATTACTTCGCTGGGAGCCATTGTGTCCTGCTACGGGAA
AACCAAGTGCACGGCCTCAAACAAGAACCGGGGTATCATTAAGACCTTCTCCAACGGCTGCGA
CTATGTGTCCAACAAGGGGGTGGACACTGTGTCCGTGGGAAACACCTTGTATTACGTGAACAA
GCAGGAGGGAAAGTCCCTCTACGTGAAGGGCGAACCCATCATCAATTTCTACGACCCGCTCGT
GTTCCCCTCCGATGAATTCGACGCATCCATCTCACAAGTCAACGAAAAGATTAACCAGTCCCTG
GCTTTCATTCGCAAGTCCGACGAACTGCTCCATAACGTCAACGCTGGAAAGTCCACCACCAAC
ATCATGATCACCACGATCATTATTGTGATCATCGTCATCCTGCTGTCACTGATAGCAGTGGGAC
TGCTCCTCTACTGCAAAGCGCGGTCGACCCCAGTGACACTCTCGAAGGACCAGCTGTCCGGG
ATCAACAACATCGCGTTTTCGAACTGA
FD2 DNA:
(SEQ ID NO: 8)
ATGGAACTCCTGATCCTGAAGGCCAATGCTATCACTACCATCCTGACTGCCGTCACCTTCTGCT
TCGCCTCCGGACAAAATATCACTGAAGAATTTTACCAAAGCACCTGTAGCGCGGTGTCCAAGG
GATACCTGAGCGCTCTGAGGACCGGATGGTACACCAGCGTGATTACCATCGAGCTGAGTAAC
ATCAAGAAGAACAAGTGCAACGGGACCGATGCTAAGGTCAAGTTGATCAAACAAGAGCTCGAC
AAGTACAAGAACGCCGTGACTGAGCTGCAGCTGCTGATGCAGTCAACTCAGGCCACCAACAA
CCGGGCCAGACGGGAACTGCCGAGATTCATGAACTACACCCTGAACAACGCCAAAAAGACCA
ACGTGACCCTGTCCAAGAAGAGAAAGCGCCGGTTCCTGGGTTTCCTGCTTGGCGTGGGATCA
GCAATCGCGTCCGGAGTGGCAGTGTCCAAGGTCTTGCACCTCGAGGGCGAAGTGAACAAGAT
CAAGTCCGCGCTTCTGTCGACCAACAAGGCCGTCGTTTCCCTGTCGAACGGAGTGTCCGTGC
TCACGAGCAAAGTGCTCGACCTGAAGAACTACATCGACAAACAGCTGCTGCCCATCGTCAACA
AGCAGAGCTGCAGCATCTCAAACATTGAAACCGTGATCGAGTTCCAGCAGAAGAACAACCGCC
TGCTCGAGATTACCAGAGAGTTTTCCGTGAACGCCGGCGTGACCACCCCGGTGTCGACCTAC
ATGCTCACAAATTCGGAACTTCTCTCCCTGATTAATGACATGCCCATTACTAACGATCAGAAAA
AGCTGATGTCGAACAATGTGCAGATTGTGCGCCAGCAGTCCTACTCCATCATGTCCATCATTAA
GGAAGAGGTCCTGGCCTACGTGGTGCAGTTGCCGCTGTACGGTGTCATCGATACCCCCTGCT
GGAAGCTCCATACTTCGCCCCTGTGTACTACCAACACCAAGGAAGGCTCCAACATCTGCCTGA
CCCGGACGGATCGCGGCTGGTACTGTGACAATGCCGGATCCGTGTCGTTCTTCCCGCAAGCG
GAGACTTGCAAAGTGCAGTCCAACCGGGTGTTCTGTGACACTATGAACTCCCTGACTCTGCCG
TCCGAAGTCAACCTCTGCAACGTGGACATTTTCAATCCAAAATACGACTGCAAGATAATGACCT
CCAAGACTGACGTGTCATCGTCCGTGATCACATCTCTGGGAGCCATTGTCTCCTGCTACGGAA
AGACTAAGTGCACCGCGTCGAACAAGAACAGGGGCATTATCAAGACCTTCAGCAACGGTTGC
GACTATGTGTCCAACAAGGGCGTGGATACCGTGTCCGTGGGCAACACCTTGTACTACGTGAAC
AAGCAGGAGGGGAAGTCCCTTTATGTGAAGGGGGAGCCAATCATTAACTTTTACGACCCCCTG
GTGTTCCCTAGCGACGAGTTCGACGCCTCAATCTCTCAAGTCAACGAAAAGATCAACCAGAGC
CTCGCCTTCATCCGCAAGTCCGATGAACTGCTGTCAGCCATTGGGGGTTACATCCCTGAGGCC
CCTCGGGACGGACAGGCATACGTCCGCAAGGACGGCGAATGGGTGCTGCTTAGCACCTTCCT
CTAA
FD3 DNA:
(SEQ ID NO: 9)
ATGGAACTGCTGATCCTCAAAGCCAACGCAATCACCACCATTCTCACCGCTGTGACCTTCTGC
TTCGCATCGGGGCAGAACATCACTGAAGAGTTTTACCAGAGCACTTGCAGCGCGGTGTCAAAG
GGTTACCTTTCCGCACTGCGGACCGGATGGTACACTTCCGTGATCACCATTGAGCTCAGCAAC
ATCAAGGAAAACAAGTGCAATGGCACCGACGCCAAGGTCAAGCTGATCAAACAAGAACTGGAC
AAGTACAAGAACGCCGTGACAGAATTGCAGCTCCTGATGGGATCCGGAAACGTCGGTCTGGG
CGGAGCCATCGCGAGTGGAGTGGCTGTGTCCAAGGTCTTGCACCTCGAGGGAGAAGTGAACA
AGATCAAGTCCGCGCTGCTGTCAACGAACAAGGCCGTGGTGTCCCTGTCTAACGGCGTCAGC
GTGCTGACGTTCAAGGTCCTGGACCTGAAGAATTACATTGACAAGCAGCTGCTGCCCATCCTC
AACAAGCAATCCTGCTCCATCTCCAACCCCGAAACCGTGATCGAGTTCCAGCAGAAGAACAAC
CGCCTGCTGGAAATTACTCGCGAGTTCTCTGTGAATGCCGGCGTGACCACCCCTGTGTCCACC
TACATGCTGACCAACTCCGAGCTTCTCTCCCTTATCAATGACATGCCTATCACGAACGACCAGA
AGAAGCTGATGTCGAACAACGTGCAGATTGTGCGGCAGCAGTCATACAGCATCATGTCGATCA
TCAAGGAAGAAGTGCTGGCGTACGTGGTGCAACTCCCGCTGTACGGCGTCATCGATACCCCG
TGCTGGAAGCTGCACACCTCGCCTTTGTGTACCACCAACACCAAGAACGGATCCAACATCTGC
TTAACCCGGACTGATCGGGGTTGGTACTGCGACAACGCCGGGAATGTTTCGTTCTTCCCACAA
GCCGAGACTTGTAAAGTGCAGTCAAACAGAGTGTTCTGTGACACCATGAACTCGAGAACCCTG
CCCAGCGAAGTGAACCTGTGTAACGTCGACATCTTTAACCCAAAATACGATTGCAAGATTATGA
CCAGCAAAACCGACGTGTCCTCCTCCGTGATAACAAGCCTGGGGGCGATTGTGTCATGCTAC
GGAAAGACTAAGTGCACCGCCTCGAACAAGAACCGCGGCATCATTAAGACTTTCTCGAATGGT
TGCGACTATGTGTCCAACAAGGGCGTGGATACTGTGTCAGTCGGGAATACTCTTTACTACGTG
AACAAGCAGGAGGGGAAAAGCCTCTACGTGAAGGGAGAGCCTATTATCAACTTTTACGATCCG
CTGGTGTTCCCGTCCGACGAATTCGACGCCAGCATCAGCCAAGTCAACGAGCTGATTAACCAG
TCCCTCGCCTTCATCAACCAATCCGACGAGCTCCTGCATAACGTGAACGCCGGAAAGTCCACC
ACCAACATCATGATCACTACTATTATCATCGTGATCATCGTCATCCTGCTGAGCCTGATTGCTG
TGGGCCTGTTGCTGTATTGCAAAGCCAGGTCCACCCCGGTCACCCTGTCGAAGGATCAGCTG
TCCGGAATCAACAACATTGCCTTCTCCAACTAA

The nucleic acid sequences for the 5′UTR and 3′UTR are recited below.

5′UTR:
(SEQ ID NO: 10)
GGACAGAUCGCCUGGAGACGCCAUCCACGCUGUUUUGACCUCCAUAGAAGA
CACCGGGACCGAUCCAGCCUCCGCGGCCGGGAACGGUGCAUUGGAACGCGG
AUUCCCCGUGCCAAGAGUGACUCACCGUCCUUGACACG_
3′UTR:
(SEQ ID NO: 11)
CGGGUGGCAUCCCUGUGACCCCUCCCCAGUGCCUCUCCUGGCCCUGGAAGU
UGCCACUCCAGUGCCCACCAGCCUUGUCCUAAUAAAAUUAAGUUGCAUC

The nucleic acid sequences for each of the full-length mRNA encoding the RSV F proteins are recited below.

FD1 mRNA:
(SEQ ID NO: 12)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGGAAUUGCUGAUCCUCAAAGCGAACGCAAUCACCACUAUC
CUCACUGCGGUCACCUUCUGCUUUGCGAGCGGACAGAACAUCACCGAAGAAUUCUACCAAU
CUACUUGCUCCGCCGUGUCCAAGGGUUACCUGUCCGCCCUGAGGACCGGAUGGUACACUU
CCGUGAUUACCAUUGAGUUGUCGAAUAUCAAGAAGAACAAGUGCAACGGAACCGAUGCUAA
GGUCAAGCUGAUCAAGCAGGAGCUGGACAAGUACAAGAAUGCUGUGACCGAGCUGCAGCUG
CUGAUGCAGUCCACUCAAGCCACCAACAAUCGCGCCCGGCGGGAACUCCCAAGGUUCAUGA
ACUACACCUUGAACAACGCCAAGAAAACGAACGUGACCCUGUCCAAGAAGCGCAAGCGCAGA
UUCCUUGGCUUCCUUCUGGGCGUCGGUAGCGCCAUCGCCUCCGGCGUGGCCGUCAGCAAG
GUCCUGCACCUCGAGGGAGAAGUCAACAAGAUUAAGAGCGCCCUGCUGUCCACCAACAAGG
CCGUGGUGUCGCUAUCAAACGGCGUCAGCGUACUGACCAGCAAAGUGCUGGAUCUCAAGAA
CUACAUUGAUAAGCAACUCCUCCCUAUCGUGAAUAAGCAGAGCUGUUCGAUUUCCAACAUC
GAGACUGUGAUUGAAUUCCAGCAGAAGAACAACCGGCUGCUGGAAAUUACCAGAGAAUUCA
GCGUGAAUGCCGGAGUCACUACCCCCGUGUCCACCUACAUGCUGACAAACUCCGAGCUGCU
GAGCCUGAUCAACGAUAUGCCGAUUACCAACGACCAGAAGAAGCUGAUGUCGAACAACGUG
CAGAUCGUGCGCCAGCAGUCCUACUCAAUCAUGUCGAUCAUCAAGGAAGAGGUCCUGGCCU
ACGUGGUGCAGCUUCCUCUGUACGGCGUGAUUGACACUCCGUGUUGGAAACUGCACACUA
GUCCCCUGUGCACUACUAACACCAAGGAGGGCAGCAAUAUCUGCCUGACUCGGACCGAUAG
AGGCUGGUACUGUGAUAACGCCGGGUCCGUGUCCUUCUUCCCGCAAGCCGAGACUUGCAA
AGUGCAGAGCAACCGGGUGUUCUGUGACACUAUGAACUCACUGACCUUGCCGAGCGAAGUC
AACCUUUGCAACGUGGACAUCUUUAACCCUAAAUACGACUGCAAGAUCAUGACCUCCAAGAC
CGACGUGUCGAGCUCAGUGAUUACUUCGCUGGGAGCCAUUGUGUCCUGCUACGGGAAAAC
CAAGUGCACGGCCUCAAACAAGAACCGGGGUAUCAUUAAGACCUUCUCCAACGGCUGCGAC
UAUGUGUCCAACAAGGGGGUGGACACUGUGUCCGUGGGAAACACCUUGUAUUACGUGAACA
AGCAGGAGGGAAAGUCCCUCUACGUGAAGGGCGAACCCAUCAUCAAUUUCUACGACCCGCU
CGUGUUCCCCUCCGAUGAAUUCGACGCAUCCAUCUCACAAGUCAACGAAAAGAUUAACCAG
UCCCUGGCUUUCAUUCGCAAGUCCGACGAACUGCUCCAUAACGUCAACGCUGGAAAGUCCA
CCACCAACAUCAUGAUCACCACGAUCAUUAUUGUGAUCAUCGUCAUCCUGCUGUCACUGAU
AGCAGUGGGACUGCUCCUCUACUGCAAAGCGCGGUCGACCCCAGUGACACUCUCGAAGGA
CCAGCUGUCCGGGAUCAACAACAUCGCGUUUUCGAACUGACGGGUGGCAUCCCUGUGACC
CCUCCCCAGUGCCUCUCCUGGCCCUGGAAGUUGCCACUCCAGUGCCCACCAGCCUUGUCC
UAAUAAAAUUAAGUUGCAUC
FD2 mRNA:
(SEQ ID NO: 13)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGGAACUCCUGAUCCUGAAGGCCAAUGCUAUCACUACCAUC
CUGACUGCCGUCACCUUCUGCUUCGCCUCCGGACAAAAUAUCACUGAAGAAUUUUACCAAA
GCACCUGUAGCGCGGUGUCCAAGGGAUACCUGAGCGCUCUGAGGACCGGAUGGUACACCA
GCGUGAUUACCAUCGAGCUGAGUAACAUCAAGAAGAACAAGUGCAACGGGACCGAUGCUAA
GGUCAAGUUGAUCAAACAAGAGCUCGACAAGUACAAGAACGCCGUGACUGAGCUGCAGCUG
CUGAUGCAGUCAACUCAGGCCACCAACAACCGGGCCAGACGGGAACUGCCGAGAUUCAUGA
ACUACACCCUGAACAACGCCAAAAAGACCAACGUGACCCUGUCCAAGAAGAGAAAGCGCCGG
UUCCUGGGUUUCCUGCUUGGCGUGGGAUCAGCAAUCGCGUCCGGAGUGGCAGUGUCCAAG
GUCUUGCACCUCGAGGGCGAAGUGAACAAGAUCAAGUCCGCGCUUCUGUCGACCAACAAGG
CCGUCGUUUCCCUGUCGAACGGAGUGUCCGUGCUCACGAGCAAAGUGCUCGACCUGAAGA
ACUACAUCGACAAACAGCUGCUGCCCAUCGUCAACAAGCAGAGCUGCAGCAUCUCAAACAUU
GAAACCGUGAUCGAGUUCCAGCAGAAGAACAACCGCCUGCUCGAGAUUACCAGAGAGUUUU
CCGUGAACGCCGGCGUGACCACCCCGGUGUCGACCUACAUGCUCACAAAUUCGGAACUUCU
CUCCCUGAUUAAUGACAUGCCCAUUACUAACGAUCAGAAAAAGCUGAUGUCGAACAAUGUG
CAGAUUGUGCGCCAGCAGUCCUACUCCAUCAUGUCCAUCAUUAAGGAAGAGGUCCUGGCCU
ACGUGGUGCAGUUGCCGCUGUACGGUGUCAUCGAUACCCCCUGCUGGAAGCUCCAUACUU
CGCCCCUGUGUACUACCAACACCAAGGAAGGCUCCAACAUCUGCCUGACCCGGACGGAUCG
CGGCUGGUACUGUGACAAUGCCGGAUCCGUGUCGUUCUUCCCGCAAGCGGAGACUUGCAA
AGUGCAGUCCAACCGGGUGUUCUGUGACACUAUGAACUCCCUGACUCUGCCGUCCGAAGU
CAACCUCUGCAACGUGGACAUUUUCAAUCCAAAAUACGACUGCAAGAUAAUGACCUCCAAGA
CUGACGUGUCAUCGUCCGUGAUCACAUCUCUGGGAGCCAUUGUCUCCUGCUACGGAAAGA
CUAAGUGCACCGCGUCGAACAAGAACAGGGGCAUUAUCAAGACCUUCAGCAACGGUUGCGA
CUAUGUGUCCAACAAGGGCGUGGAUACCGUGUCCGUGGGCAACACCUUGUACUACGUGAA
CAAGCAGGAGGGGAAGUCCCUUUAUGUGAAGGGGGAGCCAAUCAUUAACUUUUACGACCCC
CUGGUGUUCCCUAGCGACGAGUUCGACGCCUCAAUCUCUCAAGUCAACGAAAAGAUCAACC
AGAGCCUCGCCUUCAUCCGCAAGUCCGAUGAACUGCUGUCAGCCAUUGGGGGUUACAUCC
CUGAGGCCCCUCGGGACGGACAGGCAUACGUCCGCAAGGACGGCGAAUGGGUGCUGCUUA
GCACCUUCCUCUAACGGGUGGCAUCCCUGUGACCCCUCCCCAGUGCCUCUCCUGGCCCUG
GAAGUUGCCACUCCAGUGCCCACCAGCCUUGUCCUAAUAAAAUUAAGUUGCAUC
FD3 mRNA:
(SEQ ID NO: 14)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGGAACUGCUGAUCCUCAAAGCCAACGCAAUCACCACCAUU
CUCACCGCUGUGACCUUCUGCUUCGCAUCGGGGCAGAACAUCACUGAAGAGUUUUACCAGA
GCACUUGCAGCGCGGUGUCAAAGGGUUACCUUUCCGCACUGCGGACCGGAUGGUACACUU
CCGUGAUCACCAUUGAGCUCAGCAACAUCAAGGAAAACAAGUGCAAUGGCACCGACGCCAA
GGUCAAGCUGAUCAAACAAGAACUGGACAAGUACAAGAACGCCGUGACAGAAUUGCAGCUC
CUGAUGGGAUCCGGAAACGUCGGUCUGGGCGGAGCCAUCGCGAGUGGAGUGGCUGUGUC
CAAGGUCUUGCACCUCGAGGGAGAAGUGAACAAGAUCAAGUCCGCGCUGCUGUCAACGAAC
AAGGCCGUGGUGUCCCUGUCUAACGGCGUCAGCGUGCUGACGUUCAAGGUCCUGGACCUG
AAGAAUUACAUUGACAAGCAGCUGCUGCCCAUCCUCAACAAGCAAUCCUGCUCCAUCUCCAA
CCCCGAAACCGUGAUCGAGUUCCAGCAGAAGAACAACCGCCUGCUGGAAAUUACUCGCGAG
UUCUCUGUGAAUGCCGGCGUGACCACCCCUGUGUCCACCUACAUGCUGACCAACUCCGAGC
UUCUCUCCCUUAUCAAUGACAUGCCUAUCACGAACGACCAGAAGAAGCUGAUGUCGAACAA
CGUGCAGAUUGUGCGGCAGCAGUCAUACAGCAUCAUGUCGAUCAUCAAGGAAGAAGUGCUG
GCGUACGUGGUGCAACUCCCGCUGUACGGCGUCAUCGAUACCCCGUGCUGGAAGCUGCAC
ACCUCGCCUUUGUGUACCACCAACACCAAGAACGGAUCCAACAUCUGCUUAACCCGGACUG
AUCGGGGUUGGUACUGCGACAACGCCGGGAAUGUUUCGUUCUUCCCACAAGCCGAGACUU
GUAAAGUGCAGUCAAACAGAGUGUUCUGUGACACCAUGAACUCGAGAACCCUGCCCAGCGA
AGUGAACCUGUGUAACGUCGACAUCUUUAACCCAAAAUACGAUUGCAAGAUUAUGACCAGCA
AAACCGACGUGUCCUCCUCCGUGAUAACAAGCCUGGGGGCGAUUGUGUCAUGCUACGGAAA
GACUAAGUGCACCGCCUCGAACAAGAACCGCGGCAUCAUUAAGACUUUCUCGAAUGGUUGC
GACUAUGUGUCCAACAAGGGCGUGGAUACUGUGUCAGUCGGGAAUACUCUUUACUACGUGA
ACAAGCAGGAGGGGAAAAGCCUCUACGUGAAGGGAGAGCCUAUUAUCAACUUUUACGAUCC
GCUGGUGUUCCCGUCCGACGAAUUCGACGCCAGCAUCAGCCAAGUCAACGAGCUGAUUAAC
CAGUCCCUCGCCUUCAUCAACCAAUCCGACGAGCUCCUGCAUAACGUGAACGCCGGAAAGU
CCACCACCAACAUCAUGAUCACUACUAUUAUCAUCGUGAUCAUCGUCAUCCUGCUGAGCCU
GAUUGCUGUGGGCCUGUUGCUGUAUUGCAAAGCCAGGUCCACCCCGGUCACCCUGUCGAA
GGAUCAGCUGUCCGGAAUCAACAACAUUGCCUUCUCCAACUAACGGGUGGCAUCCCUGUGA
CCCCUCCCCAGUGCCUCUCCUGGCCCUGGAAGUUGCCACUCCAGUGCCCACCAGCCUUGU
CCUAAUAAAAUUAAGUUGCAUC

RSV F mRNA Protein Expression:

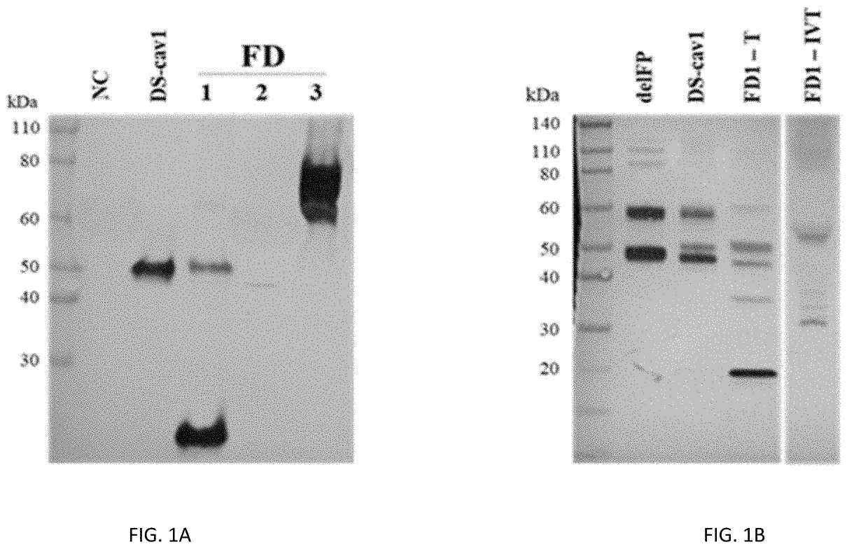

Protein expression of the FD1, FD2, and FD3 mRNA constructs was evaluated. The mRNA constructs were transfected into human embryonic kidney cells (HEK) and cell lysates or supernatants were recovered 24-hours later for analysis by western blot. FIG. 1A shows the western blot image of proteins recovered from the transfected cells and detected using the 5353C75 mouse monoclonal antibody. The FD1 construct produced a band of moderate intensity at the expected molecular weight (MVV) of ˜50 kDa based on DS-Cav1 protein control, however, there was also a very dense band at <30 kDa. The FD2 band was expressed at the correct MW but was substantially less dense than the 50 kDa band from FD1 indicating lower expression. The FD3 band was much larger and smeared from 60-80 kDa, which is consistent with FD3 being a glycosylated protein. Neither FD2 nor FD3 expressed a protein band at <30 kDa like FD1. To investigate the low molecular weight band observed with FD1, in vitro translation was performed on the mRNA. As shown in FIG. 1B, a western blot was prepared with HEK transfected proteins from FD1 mRNA in the lane marked “FD1-T” and the in vitro translation product from FD1 mRNA in the lane marked “FD1-IVT”. The lack of expression of the small MW band in the FD1-IVT lane of this blot indicates that the small MW band may be due to an issue with protein processing by the HEK cells.

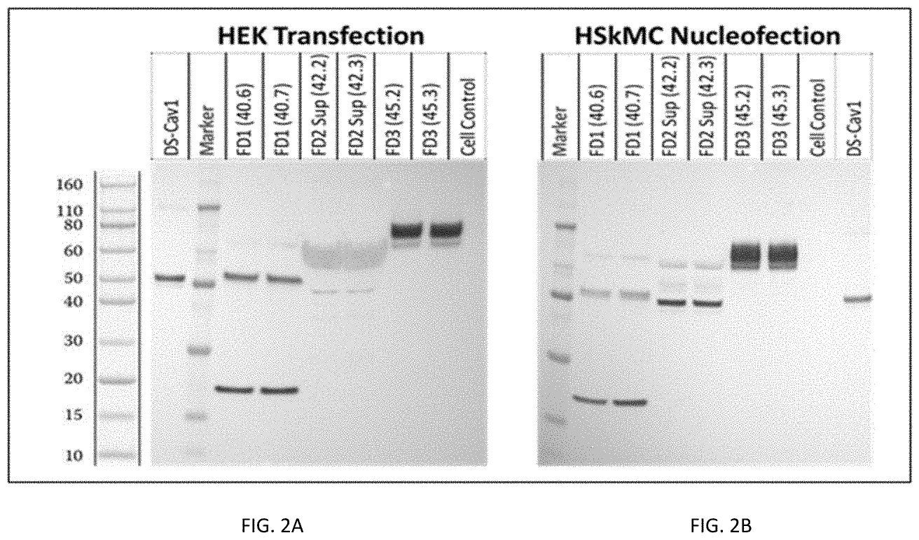

To further investigate the low molecular weight band expression by FD1 and ensure consistency between mRNA lots, two newly transcribed lots of mRNA were prepared for all three FD constructs. These mRNAs were transfected into HEK cells as well as nucleofected into human skeletal muscle cells (HSkMCs), as shown in FIG. 2A and FIG. 2B.

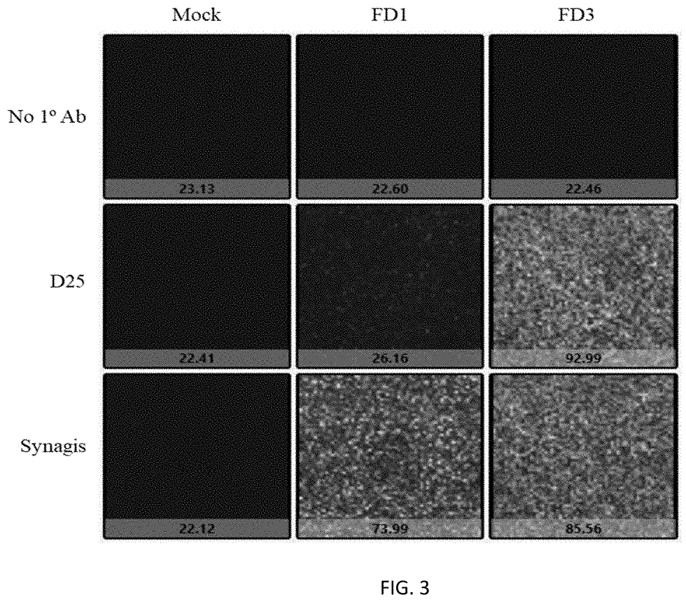

Lastly, immunostaining was performed on membrane-bound FD1 and FD3 constructs to evaluate cell surface expression and binding of monoclonal antibodies against known antigenic sites. HEK cells were transfected with FD1 or FD3 mRNA and probed with monoclonal antibodies 24-hours post transfection. As shown in FIG. 3, although both constructs had a high signal when probed with the Site II-specific monoclonal antibody, unlike the FD3 transfected cells, the FD1 construct had very little signal over background (mock transfected) when probed with the Site 0-specific monoclonal antibody D25. This indicates that the RSV-F protein being expressed on the surface of the FD1 transfected cells is not displaying the antigenic site with the highest neutralizing potential. Together with the western blots showing an unexpected banding pattern (e.g., FIG. 1A, 1B, FIG. 2A, and FIG. 2B), these data suggest that FD1 may not induce effective protection in vivo and immunogenicity testing would need to be conducted to confirm this.

### Example 2: Immunogenicity of mRNA Encoding RSV F Protein in Mice

The immunogenicity of mRNA encoding the RSV F proteins FD1, FD2, and FD3 recited above were tested in un-immunized (naïve) mice. Each mRNA was encapsulated into a lipid nanoparticle (LNP).

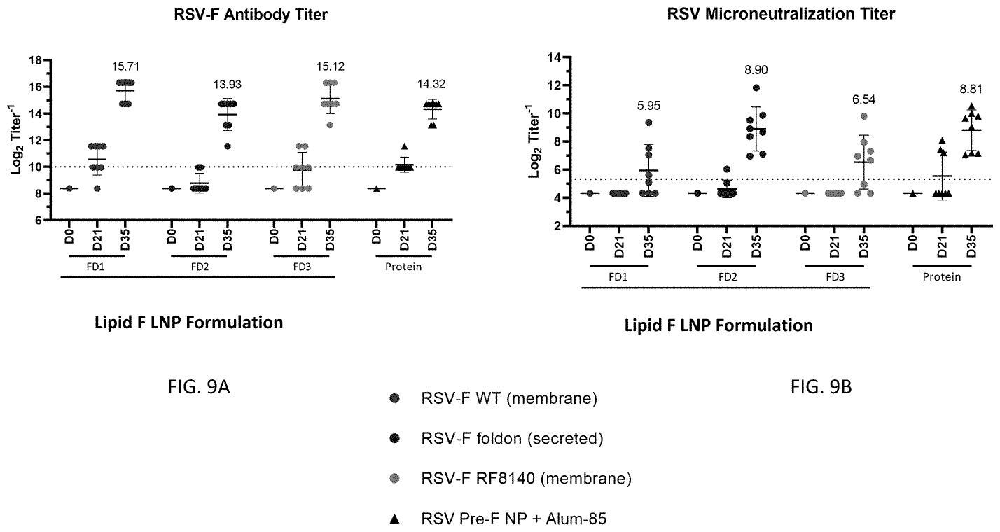

Each LNP-mRNA composition was administered to naïve mice at a dose of 1 μg per mouse. As a control, a pre-fusion F protein nanoparticle (Pre-F-NP) was administered to mice with an alum-85 adjuvant. Alum-85 (also known as alhydrogel-85) is a type of aluminum hydroxide-based adjuvant. The Pre-F-NP utilizes a ferritin domain at the C terminus of the Pre-F protein, with each ferritin domain self-assembling with other ferritin domains to form the globular nanoparticles. At day 0, 21, and 35 post-injection, serum was extracted from the immunized mice. The RSV F protein antibody titer (FIG. 9A) and RSV microneutralization titer (FIG. 9B) were measured. Antibody titer was measured as follows: ELISA plates were coated with the RSV prefusion F protein at 1 μg/ml. After blocking, plates then received a serial dilution of each serum sample covering a dilution range from 1:1000 to 1:729,000. The plates were then detected using an anti-mouse secondary antibody conjugated to horse radish peroxidase, followed by visualization using the Pierce 1-Step Ultra TMB-ELISA Substrate Solution. Plates were then read at 450 nm in SpectraMax plate reader. Titers were reported as the highest serum dilution resulting in an optical density>0.2.

The RSV microneutralization titer was measured as follows: Vero cells (ATCC CCL-81) were seeded at 30,000 cells/well in 96-well plates suitable for fluorescence reading one day prior to infection. Serum samples were heat inactivated and 4-fold serially diluted from 1:20 to 1:81,920. Diluted sera were combined 1:1 with RSV strain A2 expressing Green Florescent Protein reporter and incubated for 1 hour. The serum-virus mixtures were added to the cell plates which were incubated for 24 hours. The plates were then read on a high content imager and the florescent events were quantified. Serum 50% neutralizing titers were calculated using 4-parameter logistics regression in SoftMax GxP.

Among the FD1, FD2, and FD3 mRNA, the data shows that the FD1 and FD3 mRNA induced the highest RSV F-protein binding antibody titers, and FD2 and FD3 mRNA induced the highest RSV neutralization in mice.

### Example 3: Immunogenicity of mRNA Encoding RSV F Protein in the Modular Immune In Vitro Construct (MIMIC®) System

The MIMIC® system uses the circulating immune cells of individual donors to recapitulate each individual human immune response. The history of human exposure to RSV is represented in the immune populations of human donor cells used to develop MIMIC® B-cell Lymphoid Tissue Equivalent (LTE) modules. This module allows for a measurement of “recall” responses in circulating lymphocytes, the situation that is expected during immunization of the human population with pre-existing immunity to RSV. The MIMIC® system is further described in Higbee et al. (Altern. Lab Anim. 37: Suppl 1: 19-27. 2009), incorporated herein by reference. mRNA-LNP compositions were incubated with the MIMIC® system. Each LNP was composed of 40% cationic Lipid F, 30% phospholipid DOPE, 1.5% PEGylated lipid DMG PEG2000, and 28.5% cholesterol.

To confirm the antibody response of FD1, FD2, and FD3, a dose of 0.37 μg of mRNA-LNP was used in the MIMIC® B cell LTE. Fourteen days later, MIMIC® supernatants were collected and tested in a Luminex-based Antibody Forensics assay for antibody binding to Pre-F and Post-F.

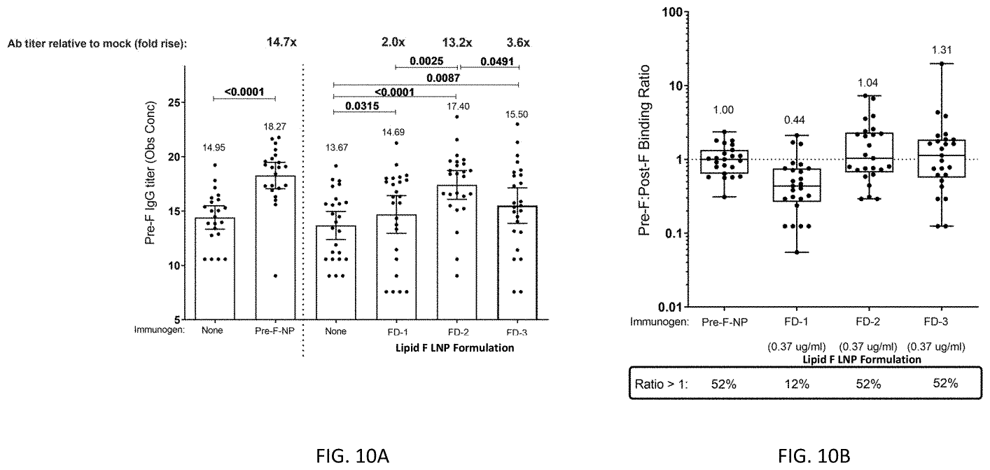

The titer of anti-Pre-F protein IgG was measured for each of FD1, FD2, and FD3 mRNA, along with Pre-F NP as a control. As shown in FIG. 10A, each of the three mRNA stimulated robust production of anti-Pre-F protein IgG. The anti-Pre-F/anti-Post-F antibody ratio was also measured. As shown in FIG. 10B, FD3 mRNA stimulated the generation of anti-Pre-F antibodies to a greater extent than FD1 or FD2 mRNA.

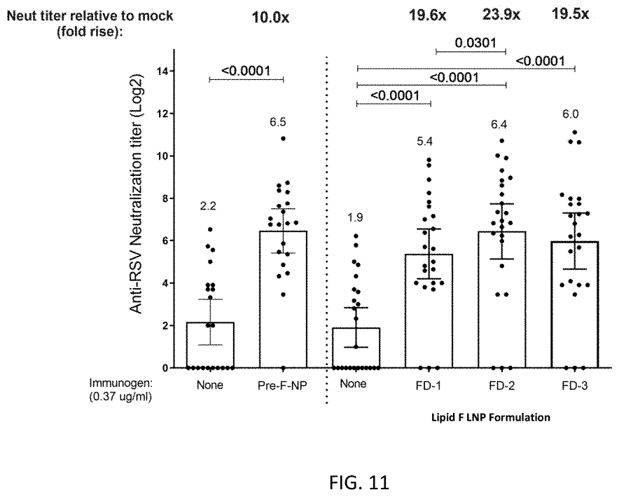

The neutralizing antibody titers induced in the MIMIC® system by the stimulations described above were determined. As shown in FIG. 11, each of FD1, FD2, and FD3 mRNA stimulated a high anti-RSV neutralization titer.

### Example 4: Immunogenicity of mRNA Encoding RSV F Protein in Non-Human Primates (NHPs)

Immunogenicity experiments were performed in NHPs. Each mRNA was encapsulated into an LNP composed of 40% cationic lipid OF-02, 30% phospholipid DOPE, 1.5% PEGylated lipid DMG PEG2000, and 28.5% cholesterol (the “Lipid A” LNP formulation).

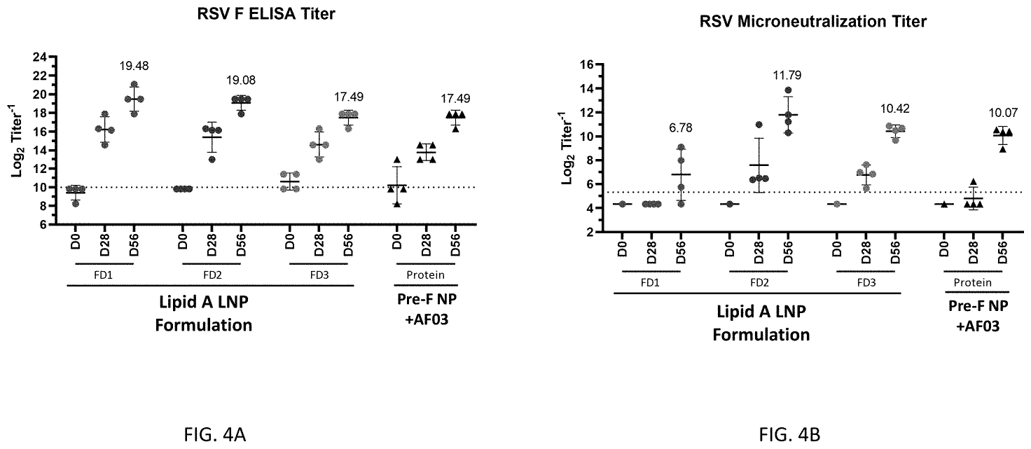

Each LNP-mRNA composition was administered to naïve NHPs at a dose of 10 μg per animal. As a control, Pre-F-NP was administered to NHPs with an AF03 adjuvant. AF03 is a squalene-based emulsion adjuvant described further in Klucker et al. (J. Pharma. Scien. 101(12): 4490-4500. 2012). At day 0, 28, and 56 post-injection, serum was extracted from the immunized NHPs. The RSV F protein antibody titer (FIG. 4A) and RSV microneutralization titer (FIG. 4B) were measured. The data shows that all three mRNA induced high RSV F-protein binding antibody titers, and FD2 and FD3 mRNA induced the highest RSV neutralization in NHPs.

Immunogenicity experiments were also performed in NHPs that were pre-immunized with Pre-F NP. Each mRNA was encapsulated into the same LNP as used above in the naïve NHP.

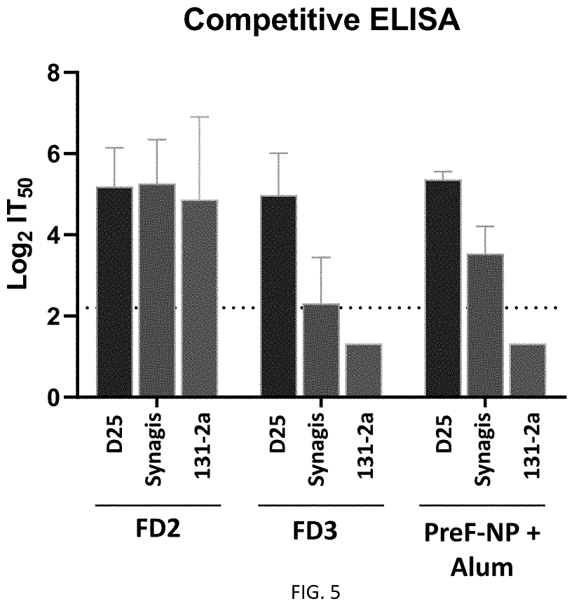

The anti-RSV F protein antibodies generated in immunized NHPs were further characterized in a competitive ELISA. The serum of immunized NHPs was used to determine the ability of the serum to compete off three different anti-RSV F protein antibodies. Antibody D25 binds to Site Ø on the Pre-F protein (McLellan et al., Science. 340(6136): 1113-7. 2013). Antibody Synagis (palivizumab) binds to Site II (Johnson et al. J. Infect. Dis. 176(5): 1215-24. 1997). Antibody 131-2a binds to Site I (Widjaja et al. J. Virol. 90(13): 5965-5977. 2016). As depicted in FIG. 5, serum from NHPs immunized with the FD3 mRNA or Pre-F NP with adjuvant had high Site Ø antibodies, as demonstrated by the high Log₂ IT₅₀ values with the D25 antibody. Moreover, limited Site I (post fusion F protein preferred, 131-2a) or Site II (Synagis) antibodies were produced. This is likely due to the modifications made to the F protein in the FD3 design, which promote the pre-fusion F protein formation and introduced glycosylation sites, which mask less productive epitopes. This allows immune refocusing on the most potent epitope in Site Ø.

In contrast to FD3, FD2 mRNA elicited similar levels of Site 0, II, and I specific antibodies. Similar results were observed in Espeseth et al. (npj Vaccines. 5(1): 16. 2020). Site II antibodies are less potent than Site Ø antibodies. Moreover, less effective antibodies from site I can potentially affect neutralizing activities or enhance infection.

### Pre-Immune NHP Evaluation:

Another factor to consider when comparing FD2 and FD3 is the ability to induce a memory B cell recall response in vaccinated individuals. Soluble proteins, such as those produced by the FD2 construct, are generally not as efficient in cross-linking the B cell receptor (BCR) and activating the memory B population as compared to membrane-bound proteins (Kowalski et al. Molecular Therapy. 27(4): 710-728. 2019; Maruggi et al. Molecular Therapy. 27(4): 757-772. 2019; Pardi et al. Nature Reviews Drug Discovery. 17(4): 261-279. 2018). Vaccination with the FD2 construct did produce a strong RSV neutralizing antibody response in naïve populations of mice and monkeys, however, the FD2 construct produces a soluble RSV-F protein and therefore should be tested in a pre-immune population to evaluate the boosting response. To test this, 12 monkeys that had previously been vaccinated with RSV Pre-F NP along with an adjuvant were used and split into 2 Groups (n=6) based on pre-immune status (by RSV-F ELISA) and previous study participation. Animals selected for the pre-immune boosting study were previously immunized with an adjuvanted RSV pre-F NP subunit vaccine. Briefly, three studies were chosen where the last immunization date was at least 6 months prior to initiation of the pre-immune boosting study. All qualifying and available animals from these three studies were screened for RSV titer by ELISA and 12 animals were selected for the pre-immune boosting study. Animals were placed in either the FD2 or FD3 boosting group based on RSV titer, sex, and previous study participation.

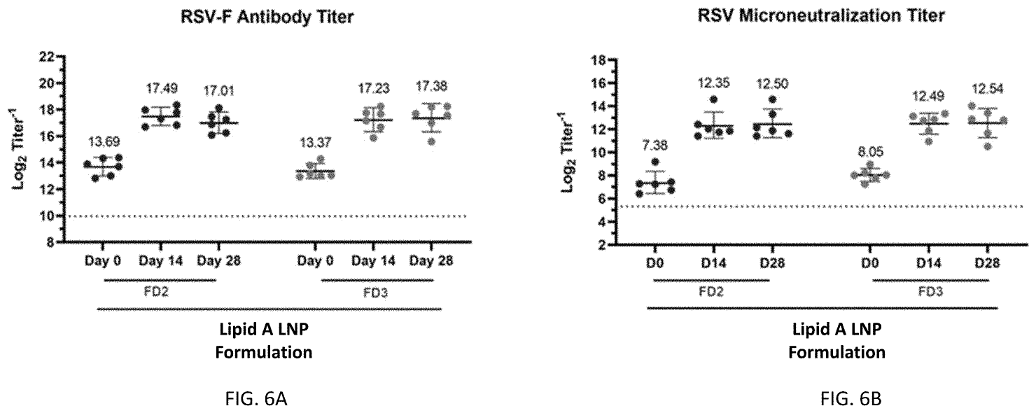

One Group was boosted with 5 μg of FD2 mRNA-LNP (Lipid A LNP formulation) and the other with 5 μg of FD3 mRNA-LNP (Lipid A LNP formulation). Blood was taken at DO, D14, and D28 and sera was evaluated by RSV-F ELISA and RSV MNA to determine the fold-rise in binding and neutralizing antibodies, respectively. Both binding antibody titers, shown in FIG. 6A, and neutralizing antibody titers, shown in FIG. 6B, significantly increased for FD2 and FD3 boosted groups from DO to D14 (p<0.001) but did not change between D14 and D28. There were no differences observed between the responses induced by FD2 and FD3 indicating that both constructs can effectively boost pre-immune individuals.

### Example 5: Cationic Lipid Screen

The effect of different cationic lipids in the LNP were tested for the LNP-encapsulated RSV F protein mRNA. Cationic lipids cKK-E10, OF-02, GL-HEPES-E3-E12-DS-4-E10, GL-HEPES-E3-E12-DS-3-E14, and GL-HEPES-E3-E10-DS-3-E18-1 were tested. Each LNP was composed of 40% of one of the five cationic lipids, 30% phospholipid DOPE, 1.5% PEGylated lipid DMG-PEG2000, and 28.5% cholesterol. An LNP with the cationic lipid MC3 was also used, considered an industry benchmark (Jayaraman et al. Angew Chem Int Ed. 51:8529-33. 2012).

Characteristics of the LNPs tested are shown below in Table 2.

TABLE 2
Characteristics of the LNPs tested

| Characteristic | Lipid A | Lipid B | Lipid C | Lipid D | Lipid E | MC3 |
|---|---|---|---|---|---|---|
| Appearance | Trans- | Trans- | Trans- | Trans- | Trans- | Trans- |
| | lucent | lucent | lucent | lucent | lucent | lucent |
| % | 93 | 99 | 88 | 90 | 94 | 98 |
| Encapsulation | | | | | | |
| Size (nm) | 89 | 101 | 103 | 87 | 74 | 89 |
| Polydispersity | 0.119 | 0.207 | 0.119 | 0.139 | 0.186 | 0.147 |
| index (PDI) | | | | | | |
| pH | 4.8 | 5.5 | 5.5 | 5.4 | 4.6 | 4.5 |

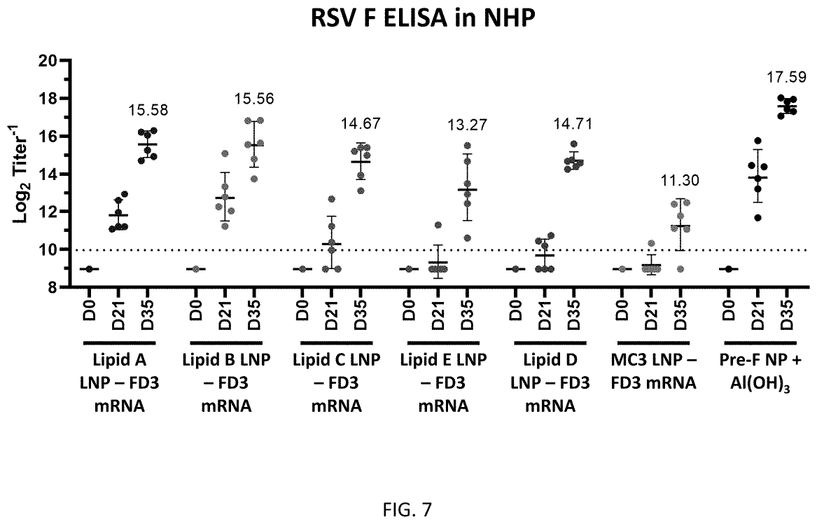

LNP-FD3 mRNA compositions were administered to NHPs. Groups of 6 cynomolgus macaques were administered a 5 μg dose of mRNA encapsulated with the above LNPs, or a 10 μg dose of an RSV Pre-F NP subunit control vaccine adjuvanted with Al(OH)₃, by intramuscular (IM) injection on DO and D21. Monkeys were bled prior to each vaccine administration as well as at two weeks post-last vaccination (D35). As shown in FIG. 7, all tested cationic lipids effectively induced the production of anti-RSV F protein antibodies to a similar level as a Pre-F NP with an aluminum adjuvant.

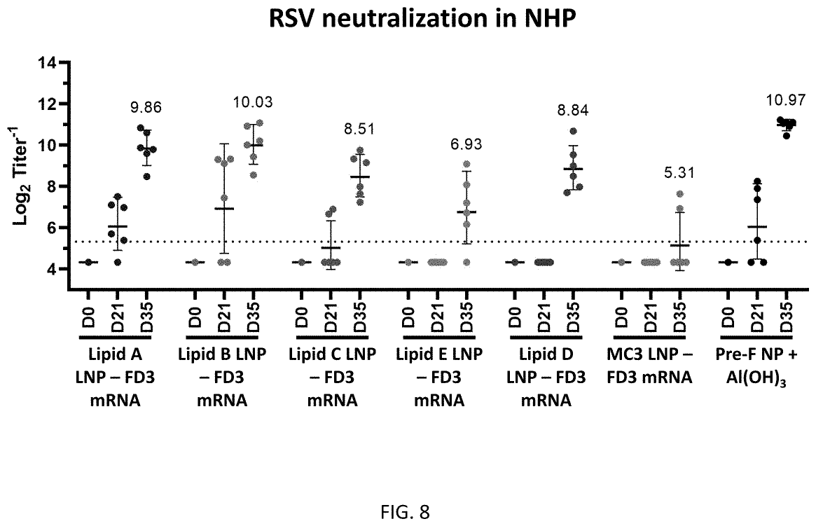

As shown in FIG. 8, all tested cationic lipids generated effective RSV neutralization titers to a similar level as a Pre-F NP with an aluminum adjuvant.

The cumulative results of FIG. 7 and FIG. 8 are shown below in Table 3 and Table 4.

TABLE 3
Magnitude of immune response

| LNP | Neutralization | |
|---|---|---|
| Formulation | Titer | Fold vs. MC3 |
| Lipid A | 9.86 | 23.43 |
| Lipid B | 10.03 | 26.35 |
| Lipid C | 8.509 | 9.18 |
| Lipid E | 6.929 | 3.07 |
| Lipid D | 8.894 | 11.99 |
| MC3 | 5.308 | 1.00 |
| Pre-F NP | 10.97 | 50.56 |

TABLE 4
Quality of immune response

| | | | Antibody Titer/ |
|---|---|---|---|
| LNP | Antibody | Neutralization | Neutralization |
| Formulation | Titer | Titer | Titer ratio |
| Lipid A | 15.58 | 9.86 | 52.71 |
| Lipid B | 15.56 | 10.03 | 46.21 |
| Lipid C | 14.67 | 8.51 | 71.51 |
| Lipid E | 13.27 | 6.93 | 81.01 |
| Lipid D | 14.71 | 8.89 | 56.49 |
| MC3 | 11.3 | 5.31 | 63.56 |
| Pre-F NP | 17.59 | 10.97 | 98.36 |

A better quality of an immune response is demonstrated with a lower value for the antibody titer/neutralization titer ratio. Here, an LNP containing cationic lipid cKK-E10 demonstrated the best quality of immune response, while all cationic lipids demonstrated a superior quality of immune response compared to the non-mRNA vaccine, Pre-F NP.

### Example 6: A Phase I/II, Randomized, Double-Blind, Placebo-Controlled Multi-Arm Dose-Finding Study to Evaluate the Safety and Immunogenicity of an RSV mRNA Vaccine Candidate Formulated with Either LNP cKK-E10 or LNP GL-HEPES-E3-E12-DS-4-E10 in Adult Participants Aged 18 to 50 Years or 60 Years of Age and Older

### Study Rationale

The clinical trial described herein tests the safety and immunogenicity of the RSV mRNA LNP vaccine. The RSV mRNA LNP vaccine comprises an RSV mRNA in one of two encapsulated LNPs formulations (i.e., an LNP containing either cKK-E10 or GL-HEPES-E3-E12-DS-4-E10) administered at three different doses (i.e., low dose, medium dose, or high dose) in healthy adults aged 18-50 years of age in the Sentinel Cohort and 60 years and older in the Main and Booster Cohorts. The RSV mRNA LNP vaccine is provided as a liquid frozen solution in a vial for intramuscular (IM) administration.

### Brief Summary

The purpose of this study is to assess the safety and immunogenicity of a single intramuscular (IM) injection of 3 dose-levels of a respiratory syncytial virus (RSV) messenger ribonucleic acid (mRNA) vaccine candidate formulated with 2 different lipid nanoparticles (LNPs) (i.e., LNP containing cKK-E10 or GL-HEPES-E3-E12-DS-4-E10) in healthy adult participants aged 18-50 years of age in the Sentinel Cohort and 60 years and older in the Main and Booster Cohorts. The primary objectives of this study are to assess the safety and immunogenicity profiles across the dose-level groups (low, medium, and high doses) with 2 LNPs (cKK-E10 and GL-HEPES-E3-E12-DS-4-E10). In addition, the study will evaluate the safety and immunogenicity of a booster vaccination administered 12 months after the primary vaccination in a subset of the study population. The duration of each participant's participation is 12 months for the Sentinel and Main Cohorts and 24 months overall for the subset of participants enrolled in the Booster Cohort.

TABLE 5
Summary of Study Arms
Number of arms: 7

| Arm Label | Arm description | Arm type |
|---|---|---|
| Group 1: Sentinel | 1 injection of RSV mRNA LNP cKK-E10vaccine | Experimental |
| and Main Cohorts | (Low dose) via intramuscular injection | |
| Group 2: Sentinel | 1 injection of RSV mRNA LNP GL-HEPES-E3- | Experimental |
| and Main Cohorts | E12-DS-4-E10vaccine (Low dose) via | |
| | intramuscular injection | |
| Group 3: Sentinel | 1 injection of RSV mRNA LNP cKK-E10vaccine | Experimental |
| and Main Cohorts | (Medium dose) via intramuscular injection | |
| Group 4: Sentinel | 1 injection of RSV mRNA LNP GL-HEPES-E3- | Experimental |
| and Main Cohorts | E12-DS-4-E10vaccine (Medium dose) via | |
| | intramuscular injection | |
| Group 5: Sentinel | 1 injection of RSV mRNA LNP cKK-E10vaccine | Experimental |
| and Main Cohorts | (High dose) via intramuscular injection | |
| Group 6: Sentinel | 1 injection of RSV mRNA LNP GL-HEPES-E3- | Experimental |
| and Main Cohorts | E12-DS-4-E10vaccine (High dose) via | |
| | intramuscular injection | |
| Group 7: Main, | 1 injection of placebo via intramuscular | Placebo |
| Sentinel and | injection | Comparator |
| Booster Cohorts | | |

### Objectives

Primary Objectives. The primary objectives are to assess the safety and immunogenicity profile of the three different dose-levels (i.e., low dose, medium dose, and high dose) of the RSV mRNA vaccine described herein encapsulated in either an LNP comprising cKK-E10 or in an LNP comprising GL-HEPES-E3-E12-DS-4-E10.

Secondary Objectives. The secondary objectives are to assess: (1) the safety profile of a booster vaccination given 12 months after the primary vaccination, in a subset of participants; (2) the durability of immune response at 3, 6, and 12 months following primary vaccination; and (3) the durability of the immune response following booster vaccination 12 months after the primary vaccination, in a subset of participants.

Endpoints. To test whether the primary and secondary objectives are met primary and secondary endpoints were established. Table 6 and Table 7 below summarize the description and time frame of evaluation of the primary and secondary endpoints, respectively.

TABLE 6
Summary of Primary Study Endpoints

| | | Time frame of |
|---|---|---|
| Endpoint title | Endpoint Description | evaluation |
| Presence of immediate | Number of participants experiencing | Within 30 minutes |
| adverse events (AEs) | immediate an immediate adverse event | after vaccination |
| Presence of solicited | Number of participants reporting: | Within 7 days after |
| injection site or | injection site reactions: pain, | vaccination |
| systemic reactions | erythema and swelling | |
| | systemic reactions: fever, headache, | |
| | malaise, myalgia, arthralgia and chills | |
| Presence of unsolicited | Number of participants experiencing | Within 28 days after |
| AEs | unsolicited AEs | vaccination |
| Presence of medically | Number of participants experiencing | Within 28 days after |
| attended adverse events | MAAEs | vaccination |
| (MAAEs) | | |
| Presence of serious | Number of participants experiencing | Month 12 |
| adverse events (SAEs) | SAEs | |
| Presence of adverse | Number of participants experiencing | Month 12 |
| events of special | AESIs | |
| interest (AESIs) | | |
| Presence of out-of- | Number of participants with | Within 7 days after |
| range biological | biological safety assessment values | vaccination |
| test results | out of normal range (as per the | |
| | laboratory performing the test) | |
| Geometric Mean Titers | Neutralizing antibody (nAb) titers | Day 1 and Day 29 |
| (GMTs) of neutralizing | post-primary vaccination | |
| antibody (nAb) titers | | |
| post-primary vaccination | | |

TABLE 7
Summary of Secondary Study Endpoints

| | | Time frame of |
|---|---|---|
| Endpoint title | Endpoint Description | evaluation |
| Presence of immediate | Number of participants experiencing | Within 30 minutes after |
| adverse events post- | immediate an immediate adverse event | vaccination |
| booster vaccination | | |
| Presence of solicited | Number of participants reporting: | Within 7 days after |
| injection site or | injection site reactions: pain, | vaccination |
| systemic reactions | erythema and swelling | |
| post-booster vaccination | systemic reactions: fever, headache, | |
| | malaise, myalgia, arthralgia and chills | |
| Presence of unsolicited | Number of participants experiencing | Within 28 days after |
| AEs post-booster vaccination | unsolicited AEs | vaccination |
| Presence of medically | Number of participants experiencing | Within 28 days after |
| attended adverse events | MAAEs | vaccination |
| post-booster vaccination | | |
| Presence of serious | Number of participants experiencing | Throughout the booster |
| adverse events (SAEs) | SAEs | study, approximately 12 |
| post-booster vaccination | | months |
| Presence of adverse events | Number of participants experiencing | Throughout the booster |
| of special interest | AESIs | study, approximately 12 |
| post-booster vaccination | | months |
| Presence of out-of-range | Number of participants with | Within 7 days after |
| biological test results | biological safety assessment | vaccination |
| post-booster vaccination | values out of normal range | |
| | (as per the laboratory | |
| | performing the test) | |
| RSV-A serum nAb titers at | RSV-A serum nAb titers at | Day 1, Day 29, Month 3, |
| pre-vaccination (D 01) and | pre-vaccination (D 01), 28 | Month 6 and Month 12 |
| 28 days post-primary | days (D 29), and 3, 6, and | |
| vaccination | 12 months post-primary vaccination | |
| GMTs of serum anti-F | Serum anti-F immunoglobulin G | Day 1, Day 29, Month 3, |
| immunoglobulin G (IgG) | (IgG) antibody (Ab) titers at | Month 6 and Month 12 |
| antibody (Ab) titers post- | pre-vaccination (D 01), 28 days | |
| primary vaccination | (D 29), and 3, 6, and 12 months | |
| | post-primary vaccination | |
| GMTs of RSV-A serum nAb | RSV-A serum nAb titers (expressed | Day 1, Day 29, Month 3, |
| post-booster vaccination | as geometric mean titers) at pre- | Month 6 and Month 12 |
| | booster vaccination, 28 days, and | post-booster |
| | 3, 6, and 12 months post-booster | |
| | vaccination | |
| GMTs of serum anti-F | Serum anti-F immunoglobulin (IgG) | Day 1, Day 29, Month 3, |
| immunoglobulin G (IgG) | antibody Ab titers (expressed as | Month 6 and Month 12 |
| antibody (Ab) titers | geometric mean titers) at pre- | post-booster |
| post-booster vaccination | booster vaccination, 28 days, | |
| | and 3, 6, and 12 months | |
| | post- booster vaccination | |

### Study Population

### Inclusion and Exclusion Criteria

Inclusion Criteria. In the Sentinel Cohort aged 18 to 50 years on the day of inclusion, in the Main and Booster Cohorts aged 60 years or older on the day of inclusion. A female participant is eligible to participate if she is not pregnant or breastfeeding and is of non-childbearing potential. To be considered of non-childbearing potential, a female must be postmenopausal for at least 1 year or surgically sterile. Able to attend all scheduled visits and to comply with all study procedures. Informed consent form has been signed and dated

Exclusion Criteria. Participants are excluded from the study if any of the following criteria apply: known or suspected congenital or acquired immunodeficiency; or receipt of immunosuppressive therapy, such as anti-cancer chemotherapy or radiation therapy, within the preceding 6 months; or long-term systemic corticosteroid therapy (prednisone or equivalent for more than 2 consecutive weeks within the past 3 months); known systemic hypersensitivity to any of the study intervention components (e.g., polyethylene glycol and polysorbate); history of a life-threatening reaction to the study interventions used in the study or to a product containing any of the same substances; any allergic reaction (e.g., anaphylaxis) after administration of mRNA COVID-19 vaccine; history of RSV-associated illness, diagnosed clinically, serologically, or microbiologically in the last 12 months; previous history of myocarditis, pericarditis, and/or myopericarditis; thrombocytopenia or bleeding disorder, contraindicating IM injection based on Investigator's judgment; bleeding disorder, or receipt of anticoagulants in the 3 weeks preceding inclusion, contraindicating intramuscular injection; chronic illness that, in the opinion of the investigator, is at a stage where it might interfere with study conduct or completion; alcohol, prescription drug, or substance abuse that, in the opinion of the Investigator, might interfere with the study conduct or completion; receipt of any vaccine other than mRNA vaccine in the 4 weeks preceding any study intervention administration or planned receipt of any vaccine other than mRNA vaccine in the 4 weeks following any study intervention administration; receipt of any mRNA vaccine in the 60 days preceding any study intervention administration or planned receipt of any mRNA vaccine in the 60 days following any study intervention administration; previous vaccination against RSV with an investigational vaccine; receipt of immune globulins, blood, or blood-derived products in the past 3 months; receipt of oral or injectable antibiotic therapy within 72 hours prior to the first blood draw; participation at the time of study enrollment (or in the 4 weeks preceding the first study intervention administration) or planned participation during the present study period in another clinical study investigating a vaccine, drug, medical device, or medical procedure; deprived of freedom by an administrative or court order, or in an emergency setting, or hospitalized involuntarily; self-reported or documented human immunodeficiency virus (HIV) detected by any FDA approved/validated test, hepatitis B virus surface antigen (HBsAg), hepatitis B core antibodies (HBcAb), hepatitis C virus antibodies (HCV Abs), or positive SARS-CoV-2 RTPCR or antigen test; or identified as an Investigator or employee of the Investigator or study center with direct involvement in the proposed study, or identified as an immediate family member (i.e., parent, spouse, and natural or adopted child) of the Investigator or employee with direct involvement in the proposed study.

### Duration per Participant

The duration of each participant's participation is 12 months for the Sentinel and Main Cohorts and 24 months overall for the subset of participants enrolled in the Booster Cohort. Participants in the Sentinel Cohort (1 intra-muscular (IM) injection) will be followed for 12 months post-vaccination. Participant in the Main Cohort (1 IM injection) will be followed for 12 months post-vaccination. Participants in the Booster Cohort (1 IM injection 12 months after the primary vaccination) will be followed for 12 months after administration of the booster dose.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

1. A respiratory syncytial virus (RSV) vaccine, comprising a messenger RNA (mRNA) comprising an open reading frame (ORF) encoding an RSV F protein antigen, wherein the RSV F protein antigen comprises an amino acid sequence with at least 98% identity to SEQ ID NO: 3 or consists of an amino acid sequence of SEQ ID NO: 3.

2. The RSV vaccine of claim 1, wherein the RSV F protein antigen is a pre-fusion protein.

3. The RSV vaccine of claim 1, wherein the ORF is codon optimized.

4. The RSV vaccine of claim 1, wherein the mRNA comprises at least one 5′ untranslated region (5′ UTR), at least one 3′ untranslated region (3′ UTR), and at least one polyadenylation (poly(A)) sequence.

5. The RSV vaccine of claim 1, wherein the mRNA comprises at least one chemical modification;
wherein at least 20%, at least 30%, at least 40%, at least 50%, at least 60%, at least 70%, at least 80%, at least 85%, at least 90%, at least 95%, or 100% of the uracil nucleotides in the mRNA are chemically modified; or
wherein at least 20%, at least 30%, at least 40%, at least 50%, at least 60%, at least 70%, at least 80%, at least 85%, at least 90%, at least 95%, or 100% of the uracil nucleotides in the ORF are chemically modified.

6-7. (canceled)

8. The RSV vaccine of claim 5, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

9-10. (canceled)

11. The RSV vaccine of claim 1, wherein the mRNA is formulated in a lipid nanoparticle (LNP).

12. The RSV vaccine of claim 11, wherein the LNP comprises at least one cationic lipid optionally wherein the LNP the cationic lipid is biodegradable, the cationic lipid is not biodegradable, the cationic lipid is cleavable, or the cationic lipid is not cleavable.

13-16. (canceled)

17. The RSV vaccine of claim 12, wherein the cationic lipid is selected from the group consisting of OF-02, cKK-E10, GL-HEPES-E3-E10-DS-3-E18-1, GL-HEPES-E3-E12-DS-4-E10, and GL-HEPES-E3-E12-DS-3-E14 optionally wherein the cationic lipid is cKK-E10 or GL-HEPES-E3-E12-DS-4-E10.

18-19. (canceled)

20. The RSV vaccine of claim 11, wherein the LNP further comprises a polyethylene glycol (PEG) conjugated (PEGylated) lipid, a cholesterol-based lipid, and a helper lipid.

21. The RSV vaccine of claim 11, wherein the LNP comprises:
a cationic lipid at a molar ratio of 35% to 55%;
a polyethylene glycol (PEG) conjugated (PEGylated) lipid at a molar ratio of 0.25% to 2.75%,
a cholesterol-based lipid at a molar ratio of 20% to 45%, and
a helper lipid at a molar ratio of 5% to 35%,
wherein all of the molar ratios are relative to the total lipid content of the LNP; or
a cationic lipid at a molar ratio of 40%,
a PEGylated lipid at a molar ratio of 1.5%,
a cholesterol-based lipid at a molar ratio of 28.5%, and
a helper lipid at a molar ratio of 30%.

22. (canceled)

23. The RSV vaccine of claim 20, wherein the PEGylated lipid is dimyristoyl-PEG2000 (DMG-PEG2000) or 2-[(polyethylene glycol)-2000]-N,N-ditetradecylacetamide (ALC-0159).

24. The RSV vaccine of claim 20, wherein the cholesterol-based lipid is cholesterol.

25. The RSV vaccine of claim 20, wherein the helper lipid is 1,2-dioleoyl-SN-glycero-3-phosphoethanolamine (DOPE) or 1,2-distearoyl-sn-glycero-3-phosphocholine (DSPC).

26. The RSV vaccine of claim 11, wherein the LNP comprises:
GL-HEPES-E3-E12-DS-4-E10 or cKK-E10 at a molar ratio of 40%,
DMG-PEG2000 at a molar ratio of 1.5%,
cholesterol at a molar ratio of 28.5%, and
DOPE at a molar ratio of 30%.

27. (canceled)

28. The RSV vaccine of claim 11, wherein the LNP has an average diameter of 30 nm to 200 nm or wherein the LNP has an average diameter of 80 nm to 150 nm.

29. (canceled)

30. The RSV vaccine of claim 1, wherein the mRNA comprises a nucleic acid sequence with at least 80% identity to a nucleic acid sequence set forth in SEQ ID NO: 6 or SEQ ID NO: 14.

31. (canceled)

32. The RSV vaccine of claim 1, wherein the mRNA comprises of the following structural elements:
(i) a 5′ cap with the following structure: 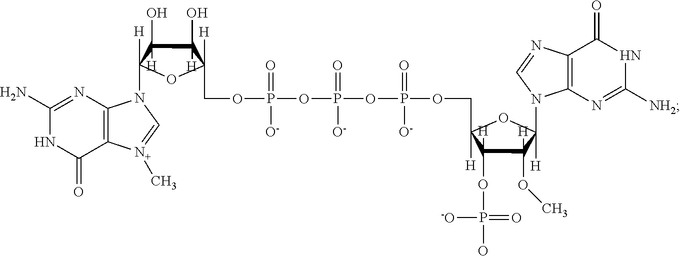
(ii) a 5′ untranslated region (5′ UTR) having the nucleic acid sequence of SEQ ID NO: 10;
(iii) a protein coding region having the nucleic acid sequence of SEQ ID NO: 6;
(iv) a 3′ untranslated region (3′ UTR) having the nucleic acid sequence of SEQ ID NO: 11; and
(v) a poly(A) tail.

33. A respiratory syncytial virus (RSV) vaccine, comprising a messenger RNA (mRNA) comprising an open reading frame (ORF) encoding an RSV F protein antigen, wherein the mRNA comprises of the following structural elements:
(i) a 5′ cap with the following structure: 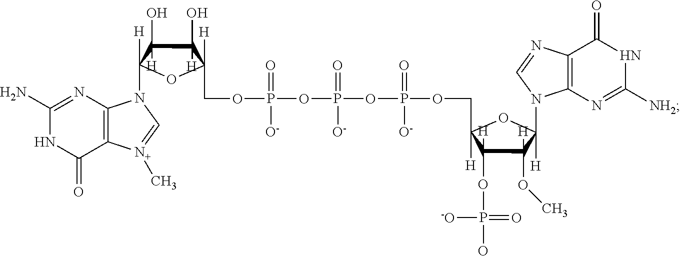
(ii) a 5′ untranslated region (5′ UTR) having the nucleic acid sequence of SEQ ID NO: 10;
(iii) a protein coding region having the nucleic acid sequence of SEQ ID NO: 6;
(iv) a 3′ untranslated region (3′ UTR) having the nucleic acid sequence of SEQ ID NO: 11; and
(v) a poly(A) tail;
wherein the mRNA is formulated in a lipid nanoparticle (LNP) comprising:
GL-HEPES-E3-E12-DS-4-E10 at a molar ratio of 40%,
DMG-PEG2000 at a molar ratio of 1.5%,
cholesterol at a molar ratio of 28.5%, and
DOPE at a molar ratio of 30%.

34. A respiratory syncytial virus (RSV) vaccine, comprising a messenger RNA (mRNA) comprising an open reading frame (ORF) encoding an RSV F protein antigen, wherein the mRNA comprises of the following structural elements:
(i) a 5′ cap with the following structure: 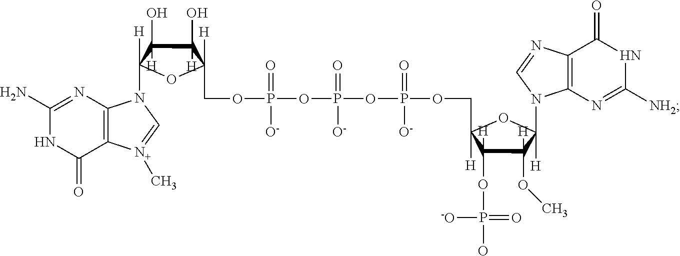
(ii) a 5′ untranslated region (5′ UTR) having the nucleic acid sequence of SEQ ID NO: 10;
(iii) a protein coding region having the nucleic acid sequence of SEQ ID NO: 6;
(iv) a 3′ untranslated region (3′ UTR) having the nucleic acid sequence of SEQ ID NO: 11; and
(v) a poly(A) tail;
wherein the mRNA is formulated in a lipid nanoparticle (LNP) comprising:
cKK-E10 at a molar ratio of 40%,
DMG-PEG2000 at a molar ratio of 1.5%,
cholesterol at a molar ratio of 28.5%, and
DOPE at a molar ratio of 30%.

35. A method of eliciting an immune response to RSV or protecting a subject against RSV infection, comprising administering the RSV vaccine of claim 1 to a subject, or wherein:
the subject has a higher serum concentration of neutralizing antibodies against RSV after administration of the RSV vaccine, relative to a subject that is administered an RSV vaccine comprising an mRNA ORF encoding an RSV F protein antigen of SEQ ID NO: 1;
the subject has a comparable serum concentration of neutralizing antibodies against RSV after administration of the RSV vaccine, relative to a subject that is administered a protein RSV vaccine;
the RSV vaccine increases the serum concentration of antibodies with binding specificity to site Ø of the RSV F protein;
the subject has a lower serum concentration of antibodies with binding specificity to site I or site II of the RSV F protein after administration of the RSV vaccine, relative to a subject that is administered an RSV vaccine comprising an mRNA ORF encoding an RSV F protein antigen of SEQ ID NO: 2; or
the RSV vaccine increases the serum concentration of neutralizing antibodies in a subject with pre-existing RSV immunity.

36-32. (canceled)

38. The method of claim 35, wherein the protein RSV vaccine is co-administered with an adjuvant.

39-43. (canceled)
